(19) United States
(12) Patent Application Publication
(10) Pub. No.: US 2005/0191614 A1
(43) Pub. Date: Sep. 1, 2005

(54) High-throughput formation, identification and analysis of diverse solid forms

(75) Inventors: Michael J. Cima, Winchester, MA (US); Douglas Levinson, Sherborn, MA (US); Anthony V. Lemmo, Sudbury, MA (US); Nicholas Galakatos, Sudbury, MA (US); David A. Putnam, Cambridge, MA (US)

Correspondence Address: Frank C. Eisenschenk, Ph.D.;Saliwanchik, Lloyd & Saliwanchik,, A Professional Association, P.O. Box 142950, Gainesville, FL, 32614-2950, US

(73) Assignee: Millenium Pharmaceuticals, Inc., Cambridge, MA (US)

(21) Appl. No.: 11/051,698

(22) Filed: Jan. 31, 2005

Related U.S. Application Data
(63) Continuation of application No. 10/372,524, filed on Feb. 21, 2003.
Application No. 10/372,524 is a continuation of application No. 09/756,092, filed on Jan. 8, 2001.
(60) Provisional application No. 60/221,539, filed on Jul. 28, 2000.
(60) Provisional application No. 60/196,821, filed on Apr. 13, 2000.
(60) Provisional application No. 60/175,047, filed on Jan. 7, 2000.

(51) Int. Cl. C12Q 1/00; G06F 19/00; G01N 33/48; G01N 33/50

(52) U.S. Cl. 435/4; 702/19

(57) ABSTRACT

The invention concerns arrays of solid-forms of substances, such as compounds and rapid-screening methods therefor to identify solid-forms, particularly of pharmaceuticals, with enhanced properties. Such properties include improved bioavailability, solubility, stability, delivery, and processing and manufacturing characteristics. The invention relates to a practical and cost-effective method to rapidly screen hundreds to thousands of samples in parallel. The invention further provides methods for determining the conditions and/or ranges of conditions required to produce crystals with desired compositions, particle sizes, habits, or polymorphic forms. In a further aspect, the invention provides high-throughput methods to identify sets of conditions and/or combinations of components compatible with particular solid-forms, for example, conditions and/or components that are compatible with advantageous polymorphs of a particular pharmaceutical.

This application claims the benefit of U.S. Provisional Patent Application Nos. 60/175,047 filed Jan. 7, 2000; 60/196,821filed Apr. 13, 2000; and 60/221,539 filed Jul. 28, 2000, all of which provisional applications are incorporated herein by reference in their entirety.

### 1. FIELD OF THE INVENTION

This invention is directed to the generation and processing of data derived from large numbers of samples, the samples comprising crystalline, amorphous, and other forms of solid substances, including chemical compounds. More specifically, the invention is directed to methods and systems for rapidly producing and screening large numbers of samples to detect the presence or absence of solid-forms. The invention is suited for discovering: (1) new solid-forms with beneficial properties and conditions for their formation, (2) conditions and/or compositions affecting the structural and/or chemical stability of solid-forms, (3)conditions and/or compositions that inhibit the formation of solid-forms; and (4) conditions and/or compositions that promote dissolution of solid-forms.

### 2. BACKGROUND OF THE INVENTION

2.1 Structure-Property Relationships in Solids

Structure plays an important role in determining the properties of substances. The properties of many compounds can be modified by structural changes, for example, different polymorphs of the same pharmaceutical compound can have different therapeutic activities. Understanding structure-property relationships is crucial in efforts to maximize the desirable properties of substances, such as the therapeutic effectiveness of a pharmaceutical.

2.1.1 Crystallization

The process of crystallization is one of ordering. During this process, randomly organized molecules in a solution, a melt, or the gas phase take up regular positions in the solid. The regular organization of the solid is responsible for many of the unique properties of crystals, including the diffraction of x-rays, defined melting point, and sharp, well-defined crystal faces. The term precipitation is usually reserved for formation of amorphous substances that have no symmetry or ordering and cannot be defined by habits or as polymorphs.

Both crystallization and precipitation result from the inability of a solution to fully dissolve the substance and can be induced by changing the state (varying parameters) of the system in some way. Common parameters that can be controlled to promote or discourage precipitation or crystallization include, but are not limited to, adjusting the temperature; adjusting the time; adjusting the pH; adjusting the amount or the concentration of the compound-of-interest; adjusting the amount or the concentration of a component; component identity (adding one or more additional components); adjusting the solvent removal rate; introducing of a nucleation event; introducing of a precipitation event; controlling evaporation of the solvent (e.g., adjusting a value of pressure or adjusting the evaporative surface area); and adjusting the solvent composition.

Important processes in crystallization are nucleation, growth kinetics, interfacial phenomena, agglomeration, and breakage. Nucleation results when the phase-transition energy barrier is overcome, thereby allowing a particle to form from a supersaturated solution. Growth is the enlargement of particles caused by deposition of solid substance on an existing surface. The relative rate of nucleation and growth determine the size distribution. Agglomeration is the formation of larger particles through two or more particles (e.g., crystals) sticking together. The thermodynamic driving force for both nucleation and growth is supersaturation, which is defined as the deviation from thermodynamic equilibrium.

Substances, such as pharmaceutical compounds can assume many different crystal forms and sizes. Particular emphasis has been put on these crystal characteristics in the pharmaceutical industry—especially polymorphic form, crystal size, crystal habit, and crystal-size distribution—since crystal structure and size can affect manufacturing, formulation, and pharmacokinetics, including bioavailability. There are four broad classes by which crystals of a given compound may differ: composition; habit; polymorphic form; and crystal size.

2.1.1.1 Resolution of Enantiomers by Direct Crystallization

Chiral chemical compounds that exhibit conglomerate behavior can be resolved into enantiomers by crystallization (i.e., spontaneous resolution, see e.g., Collins G. et al., Chirality in Industry, John Wiley & Sons, New York, (1992); Jacques, J. et al. Enantiomers, Racemates, and Resolutions, Wiley-Interscience, New York (1981)). Conglomerate behavior means that under certain crystallization conditions, optically-pure, discrete crystals or crystal clusters of both enantiomers will form, although, in bulk, the conglomerate is optically neutral. Thus, upon spontaneous crystallization of a chiral compound as its conglomerate, the resulting clusters of optically-pure enantiomer crystals can be mechanically separated. More conveniently, compounds that exhibit conglomerate behavior can be enantiomerically resolved by preferential crystallization, thereby obviating the need for mechanical separation. To determine whether a compound exhibits conglomerate behavior, many conditions and crystallizing mediums must be tested to find suitable conditions, such as time, temperature, solvent mixtures, and additives, etc. Once the ability of a compound to form a conglomerate has been established, direct crystallization in bulk can be effected in a variety of ways, for example, preferential crystallization. Preferential crystallization refers to crystallizing one enantiomer of a compound from a racemic mixture by inoculating a supersaturated solution of the racemate with seed crystals of the desired enantiomer. Thereafter, crystals of the optically enriched seeded enantiomer deposit. It must be emphasized the preferential crystallization works only for substances existing as conglomerates (Inagaki (1977), Chem. Pharm. Bull. 25:2497). Additives can promote preferential crystallization. There are numerous reports in which crystallization of optically active materials has been encouraged by the use foreign seed crystals (Eliel et al., Stereochemistry of Organic Compounds, John Wiley & Sons, Inc., New York (1994)). For example, insoluble additives favor the growth of crystals that are isomorphous with the seed, in contrast, the effect of soluble additives is the opposite (Jacques, J. et al. Enantiomers, Racemates, and Resolutions, Wiley-Interscience, New York (1981), p. 245). The definitive rationalization is that adsorption of the additive on the surface of growing crystals of one of the solute enantiomers hinders its crystallization while the other enantiomer crystalizes normally (Addadi et al., (1981), J. Am. Chem. Soc. 103:1249; Addadi et al., (1986) Top. Stereochem. 16:1). Methods for rapid, high-throughput screening of the many relevant variables for discovery of conditions and additives that promote resolution of chiral compounds is needed. Especially, in the pharmaceutical industry, where for example, one enantiomer of a particular pharmaceutical may be therapeutically active while the other may be less active, non-active, or toxic.

2.1.1.2 Resolution of Enantiomers Via Crystallization of Diastereomers

Enantiomeric resolution of a racemic mixture of a chiral compound can be effected by: (1) conversion into a diastereomeric pair by treatment with an enantiomerically pure chiral substance, (2) preferential crystallization of one diastereomer over the other, followed by (3) conversion of the resolved diastereomer into the optically-active enantiomer. Neutral compounds can be converted in diastereomeric pairs by direct synthesis or by forming inclusions, while acidic and basic compounds can be converted into diastereomeric salts. (For a review see Eliel et al., Stereochemistry of Organic Compounds, John Wiley & Sons, Inc., New York (1994), pp. 322-371). For a particular chiral compound, the number of reagents and conditions available for formation of diastereomeric pairs are extremely numerous. In one aspect, the optimal diastereomeric pair must be ascertained. This may involve testing hundreds of reagents to form salts, reaction products, charge transfer complexes, or inclusions with the compound-of-interest. A second aspect involves determining optimal conditions for resolution of the optimal diastereomeric pair, for example, optimal solvent mixtures, additives, times, and temperatures, etc. Standard mix and try methods that have been used in the past are impractical and optimal conditions and additives are rarely established. Thus, methods for rapid, high-throughput screening of the many relevant variables is needed.

2.1.2 Composition

Composition refers whether the solid-form is a single compound or is a mixture of compounds. For example, solid-forms can be present in their neutral form, e.g., the free base of a compound having a basic nitrogen or as a salt, e.g., the hydrochloride salt of a basic nitrogen-containing compound. Composition also refers to crystals containing adduct molecules. During crystallization or precipitation an adduct molecule (e.g., a solvent or water) can be incorporated into the matrix, adsorbed on the surface, or trapped within the particle or crystal. Such compositions are referred to as inclusions, such as hydrates (water molecule incorporated in the matrix) and solvates (solvent trapped within a matrix). Whether a crystal forms as an inclusion can have a profound effect on the properties, such as the bioavailability or ease of processing or manufacture of a pharmaceutical. For example, inclusions may dissolve more or less readily or have different mechanical properties or strength than the corresponding non-inclusion compounds.

2.1.3 Habit

The same compound can crystallize in different external shapes depending on, amongst others, the composition of the crystallizing medium. These crystal-face shapes are described as the crystal habit. Such information is important because the crystal habit has a large influence on the crystal's surface-to-volume ratio. Although crystal habits have the same internal structure and thus have identical single crystal- and powder-diffraction patterns, they can still exhibit different pharmaceutical properties (Haleblian 1975, J. Pharm. Sci., 64:1969). Thus discovering conditions or pharmaceuticals that affect crystal habit are needed.

Crystal habit can influence several pharmaceutical characteristics, for instance, mechanical factors, such as syingeability (e.g., a suspension of plate-shaped crystals can be injected through a small-bore syringe needle with greater ease than one of needle-shaped crystals), tableting behavior, filtration, drying, and mixing with other substances (e.g., excipients) and non-mechanical factors such as dissolution rate.

2.1.4 Polymorphism

Additionally, the same compound can crystallize as more than one distinct crystalline species (i.e., having a different internal structure) or shift from one crystalline species to another. This phenomena is known as polymorphism, and the distinct species are known as polymorphs. Polymorphs can exhibit different optical properties, melting points, solubilities, chemical reactivities, dissolution rates, and different bioavailabilities. It is well known that different polymorphs of the same pharmaceutical can have different pharmacokinetics, for example, one polymorph can be absorbed more readily than its counterpart. In the extreme, only one polymorphic form of a given pharmaceutical may be suitable for disease treatment. Thus, the discovery and development of novel or beneficial polymorphs is extremely important, especially in the pharmaceutical area.

2.1.5 Amorphous Solids

Amorphous solids, on the other hand, have no crystal shape and cannot be characterized according to habit or polymorphic form. A common amorphous solid is glass in which the atoms and molecules exist in a nonuniform array. Amorphous solids are usually the result of rapid solidification and can be conveniently identified (but not characterized) by x-ray powder diffraction, since these solids give very diffuse lines or no crystal diffraction pattern.

While amorphous solids may often have desirable pharmaceutical properties such as rapid dissolution rates, they are not usually marketed because of their physical and/or chemical instability. An amorphous solid is in a high-energy structural state relative to its rystalline form and thus it may crystallize during storage or shipping. Or an amorphous solid may be more sensitive to oxidation (Pikal et al., 1997, J. Pharm. Sci. 66:1312). In some cases, however, amorphous forms are desirable. An excellent example is novobiocin. Nvobiocin exists in a crystalline and an amorphous form. The crystalline form is poorly absorbed and does not provide therapeutically active blood levels, in contrast, the amorphous form is readily absorbed and is therapeutically active.

2.1.6 Particle and Crystal Size

Particulate matter, produced by precipitation of amorphous particles or crystallization, has a distribution of sizes that varies in a definite way throughout the size range. Particle and crystal size distribution is most commonly expressed as a population distribution relating to the number of particles at each size. Particle and crystal size distribution determines several important processing and product properties including particle appearance, separation of particles and crystals from the solvent, reactions, dissolution, and other processes and properties involving surface area. Control of particle and crystal size is very important in pharmaceutical compounds. The most favored size distribution is one that is monodisperse, i.e., all the crystals or particles are about the same size, so that dissolution and uptake in the body is known and reproducible. Furthermore, small particles or crystals are often preferred. The smaller the size, the higher the surface-to-volume ratio. The production of nanoparticles or nanocrystal forms of pharmaceuticals has become increasingly important. Reports indicate improved bioavailability due to either the known increase in solubility of fine particles or possible alternative uptake mechanisms that involve direct introduction of nanoparticles or nanocrystals into cells. Conventional preparation of these fine particles or crystals is based on mechanical milling of the pharmaceutical solid. The methods used include milling in a liquid vehicle and air-jet milling. Unfortunately, mechanical attrition of pharmaceutical solids is known to cause amorphization of the crystal structure. The degree of amorphization is difficult to control and scale-up performance is difficult to predict. But if methods for production of nanoparticles directly from the medium by control of processing parameters can be discovered, the added expense of milling could be obviated.

2.2 Generation of Solid-Forms

Crystallization and precipitation are phase changes that results in the formation of a crystalline solid from a solution or an amorphous solid. Crystallization also includes polymorphic shift from one crystalline species to another. The most common type of crystallization is crystallization from solution, in which a substance is dissolved at an appropriate temperature in a solvent, then the system is processed to achieve supersaturation followed by nucleation and growth. Common processing parameters include, but are not limited to, adjusting the temperature; adjusting the time; adjusting the pH; adjusting the amount or the concentration of the compound-of-interest; adjusting the amount or the concentration of a component; component identity (adding one or more additional components); adjusting the solvent removal rate; introducing of a nucleation event; introducing of a precipitation event; controlling evaporation of the solvent (e.g., adjusting a value of pressure or adjusting the evaporative surface area); and adjusting the solvent composition. Other crystallization methods include sublimation, vapor diffusion, desolvation of crystalline solvates, and grinding (Guillory, J. K., Polymorphism in Pharmaceutical Solids, 186, 1999).

Amorphous solids can be obtained by solidifying in such a way as to avoid the thermodynamically preferred crystallization process. They can also be prepared by disrupting an existing crystal structure.

Despite the development and research of crystallization methods, control over crystallization based on structural understanding and our ability to design crystals and other solid-forms are still limited. The control on nucleation, growth, dissolution, and morphology of molecular crystals remains primarily a matter of “mix and try” (Weissbuch, I., Lahav, M., and Leiserowitz, L., Molecular Modeling Applications in Crystallization, 166, 1999).

Because many variables influence crystallization, precipitation, and phase shift, and the solid-forms produced therefrom and because so many reagents and process variables are available, testing of individual solid-formation and crystal structure modification is an extremely tedious process. At present, industry does not have the time or resources to test hundreds of thousands of combinations to achieve an optimized solid-forms. At the current state of the art, it is more cost effective to use non-optimized or semi-optimized solid-forms in pharmaceutical and other formulations. To remedy these deficiencies, methods for rapid producing and screening of diverse sets of solid-forms on the order of thousands to hundreds of thousands of samples per day, cost effectively, are needed.

Despite the importance of crystal structure in the pharmaceutical industry, optimal crystal structures or optimal amorphous solids are not vigorously or systematically sought. Instead, the general trend is to develop the single solid-form that is first observed. Such lack of effort can lead to the failure of a drug candidate even though the candidate may be therapeutically useful in another solid-form, such as another polymorphic form. The invention disclosed herein addresses the issues discussed above.

### 3. SUMMARY OF THE INVENTION

In one embodiment, the invention relates to arrays comprising 2 or more samples, for example, about 24, 48, 96, to hundreds, thousands, ten thousands, to hundreds of thousands or more samples, one or more of the samples comprising solid-forms in gram, milligram, microgram, or nanogram quantities and practical and cost-effective methods to rapidly produce and screen such samples in parallel. These methods provide an extremely powerful tool for the rapid and systematic analysis, optimization, selection, or discovery of conditions, compounds, or compositions that induce, inhibit, prevent, or reverse formation of solid-forms. For example, the invention provides methods for systematic analysis, optimization, selection, or discovery of novel or otherwise beneficial solid-forms (e.g., beneficial pharmaceutical solid-forms having desired properties, such as improved bioavailability, solubility, stability, delivery, or processing and manufacturing characteristics) and conditions for formation thereof. The invention can also be used to identify those conditions where high-surface-area crystals or amorphous solids are prepared (e.g., nanoparticles) directly by precipitation or crystallization thus obviating the step of milling.

In another embodiment, the invention is useful to discover solid forms that posses preferred dissolution properties. In this embodiment, arrays of solid forms of the compound-of-interest are prepared. Each element of the array is prepared from different solvent and additive combinations with differing process histories. The solids are separated form any liquid that may be present. In this way, one has obtained an array of solid forms of the compound-of-interest. One then adds, to each sample of the array, the same dissolution medium of interest. Thus, one would add simulated gastric fluid if the application if to optimize the dissolution of drug substance in oral dosage forms. The dissolution medium of each array element is then sampled versus time to determine the dissolution profile of each solid form. Optimum solid forms are ones where dissolution is rapid and/or that the resulting solution is sufficiently metastable so as to be useful. Alternatively, one may be interested in solid forms that dissolve at a specified rate. Examination of the multitude of dissolution profiles will lead to the optimum solid form.

In a further embodiment, the invention discussed herein provides high-throughput methods to identify sets of conditions and/or combinations of components compatible with particular solid-forms, for example, conditions and/or components that are compatible with advantageous polymorphs of a particular pharmaceutical. As used herein “compatible” means that under the sets of conditions or in the presence of the combinations of components, the solid-form maintains its function and relevant properties, such as structural and chemical integrity. Compatibility also means sets of conditions or combinations of components that are more practical, economical, or otherwise more attractive to produce or manufacture a solid-form. Such conditions are important in manufacture, storage, and shipment of solid-forms. For example, a pharmaceutical manufacturer may want to test the stability of a particular polymorph of a drug under a multitude of different conditions. Such methods are suitable for applications such as determining the limits of a particular solid-form's structural or chemical stability under conditions of atmosphere (oxygen), temperature; time; pH; the amount or concentration of the compound-of-interest; the amount or concentration of one or more of the components; additional components; various means of nucleation; various means of introducing a precipitation event; the best method to control the evaporation of one or more of the components; or a combination thereof.

In another aspect, the invention described herein provides methods to test sets of conditions and components compatible to produce a particular solid-form, such as a particular polymorph of a drug. For example, a pharmaceutical manufacturer may know the optimal solid form of a particular pharmaceutical but not the optimal production conditions. The invention provides high-throughput methods to test various conditions that will produce a particular solid-form, such as temperature; time; pH; the amount or concentration of the compound-of-interest; the amount or concentration of one or more of the components; additional components; various means of nucleation; various means of introducing a precipitation event; the best method to control the evaporation of one or more of the components; or a combination thereof. Once a multitude of suitable sets of conditions are found, a determination can be made, depending on the compound-of-interest's identity and other relevant considerations and criteria the optimal conditions or conditions for scale-up testing.

In another embodiment, the invention concerns methods for the identification of conditions and/or compositions affecting the structural and/or chemical stability of solid-forms, for example, conditions or compositions that promote or inhibit polymorphic shift of a crystalline solid or precipitation of an amorphous solid. The invention also encompasses methods for the discovery of conditions and/or compositions that inhibit formation of solid-forms. The invention further encompasses methods for the discovery of conditions and/or compositions that promote dissolution of solid-forms.

In one embodiment, seed crystals of desired crystal forms can be harvested from the arrays of the invention. Such seed crystals can provided manufactures, such as pharmaceutical manufacturers, with the means to produce optimal crystal forms of compounds in commercial scale crystallizations. In another embodiment, the invention provides conditions for scale-up of bulk crystallizations in crystallizers, for example, conditions to prevent crystal agglomeration in the crystallizer.

The compound-of-interests to be screened can be any useful solid compound including, but not limited to, pharmaceuticals, dietary supplements, nutraceuticals, agrochemicals, or alternative medicines. The invention is particularly well-suited for screening solid-forms of a single low-molecular-weight organic molecules. Thus, the invention encompasses arrays of diverse solid-forms of a single low-molecular-weight molecule.

In one embodiment, the invention relates to an array of samples comprising a plurality of solid-forms of a single compound-of-interest, each sample comprising the compound-of-interest, wherein said compound-of-interest is a small molecule, and at least two samples comprise solid-forms of the compound-of-interest each of the two solid-forms having a different physical state from the other.

In another embodiment, the invention concerns an array comprising at least 24 samples each sample comprising a compound-of-interest and at least one component, wherein:
(a) an amount of the compound-of-interest in each sample is less than about 1 gram; and
(b) at least one of the samples comprises a solid-form of the compound-of-interest.

In still another embodiment, the invention relates to a method of preparing an array of multiple solid-forms of a compound-of-interest comprising:
(a) preparing at least 24 samples each sample comprising the compound-of-interest and at least one component, wherein an amount of the compound-of-interest in each sample is less than about 1 gram; and
(b) processing at least 24 of the samples to generate and array comprising at least two solid-forms of the compound-of-interest.

In still another embodiment, the invention provides a method of screening a plurality of solid-forms of a compound-of-interest, comprising:
(a) preparing at least 24 samples each sample comprising the compound-of-interest and one or more components, wherein an amount of the compound-of-interest in each sample is less than about 1 gram;
(b) processing at least 24 of the samples to generate an array wherein at least two of the processed samples comprise a solid-form of the compound-of-interest; and
(c) analyzing the processed samples to detect at least one solid-form.

In another embodiment, the invention concerns a method of identifying optimal solid-forms of a compound-of-interest, comprising:
(a) selecting at least one solid-form of the compound-of-interest present in an array comprising at least 24 samples each sample comprising the compound-of-interest and at least one component, wherein an amount of the compound-of-interest in each sample is less than about 1 gram; and
(b) analyzing the solid-form.

In still yet another embodiment, the invention provides a method to determine sets of conditions and/or components to produce particular solid-forms of a compound-of-interest, comprising:
(a) preparing at least 24 samples each sample comprising the compound-of-interest and one or more components, wherein an amount of the compound-of-interest in each sample is less than about 1 gram;
(b) processing at least 24 of the samples to generate an array wherein at least one of the processed samples comprises a solid-form of the compound-of-interest; and
(c) selecting samples having the solid-forms in order to identify the sets of conditions and/or components.

In a further embodiment, the invention concerns a method of screening conditions and/or components for compatibility with one or more selected solid-forms of a compound-of-interest, comprising:
(a) preparing at least 24 samples each sample comprising the compound-of-interest in solid or dissolved form and one or more components, wherein an amount of the compound-of-interest in each sample is less than about 1 gram;
(b) processing at least 24 of the samples to generate an array of said selected solid-forms; and
(c) analyzing the array.

In another embodiment still, the invention relates to a system to identify optimal solid-forms of a compound-of-interest, comprising:
(a) an automated distribution mechanism effective to prepare at least 24 samples, each sample comprising the compound-of-interest and one or more components, wherein an amount of the compound-of-interest in each sample is less than about 1 gram;
(b) an system effective to process the samples to generate an array comprising at least one solid-form of the compound-of-interest; and
(c) a detector to detect the solid-form.

In another embodiment, the invention relates to a method to determine a set of processing parameters and/or components to inhibit the formation of a solid-form of a compound-of-interest, comprising:
(a) preparing at least 24 samples each sample comprising a solution of the compound-of-interest and one or more components, wherein an amount of the compound-of-interest in each sample is less than about 1 gram;
(b) processing at least 24 of the samples under a set of processing parameters; and
(c) selecting the processed samples not having the solid-form to identify the set of processing parameters and/or components.

In a further embodiment, the invention concerns a method to determine a set of conditions and/or components to produce a compound-of-interest or a diastereomeric derivative thereof in stereomerically enriched or conglomerate form, comprising:
(a) preparing at least 24 samples each sample comprising the compound-of-interest or a diastereomeric derivative thereof and one or more components, wherein an amount of the compound-of-interest or the diastereomeric derivative in each sample is less than about 1 gram;
(b) processing at least 24 of the samples to generate an array wherein at least one of the processed samples comprises the compound-of-interest or the diastereomeric derivative in stereomerically enriched or conglomerate form; and
(c) selecting the stereomerically enriched or conglomerate samples in order to identify the set of conditions and/or components.

The arrays, systems, and methods of the invention are suitable for use with small amounts of the compound-of-interest and other components, for example, less than about 100 milligrams, less than about 100 micrograms, or even less than about 100 nanograms of the compound-of-interest or other components.

These and other features, aspects, and advantages of the invention will become better understood with reference to the following detailed description, examples, and appended claims.

### 4. DEFINITIONS

4.1 Array

As used herein, the term “array” means a plurality of samples, preferably, at least 24 samples each sample comprising a compound-of-interest and at least one component, wherein:
(a) an amount of the compound-of-interest in each sample is less than about 100 micrograms; and
(b) at least one of the samples comprises a solid-form of the compound-of-interest.
   Preferably, each sample comprises a solvent as a component. The samples are associated under a common experiment designed to identify solid-forms of the compound-of-interest with new and enhanced properties and their formation; to determine compounds or compositions that inhibition formation of solids or a particular solid-form; or to physically or structurally stabilize a particular solid-form, such as preventing polymorphic shift. An array can comprise 2 or more samples, for example, 24, 36, 48, 96, or more samples, preferably 1000 or more samples, more preferably, 10,000 or more samples. An array can comprise one or more groups of samples also known as sub-arrays. For example, a group can be a 96-tube plate of sample tubes or a 96-well plate of sample wells in an array consisting of 100 or more plates. Each sample or selected samples or each sample group of selected sample groups in the array can be subjected to the same or different processing parameters; each sample or sample group can have different components or concentrations of components; or both to induce, inhibit, prevent, or reverse formation of solid-forms of the compound-of-interest.

Arrays can be prepared by preparing a plurality of samples, each sample comprising a compound-of-interest and one or more components, then processing the samples to induce, inhibit, prevent, or reverse formation of solid-forms of the compound-of-interest. Preferably, the sample includes a solvent.

4.2 Sample

As used herein, the term “sample” means a mixture of a compound-of-interest and one or more additional components to be subjected to various processing parameters and then screened to detect the presence or absence of solid-forms, preferably, to detect desired solid-forms with new or enhanced properties. In addition to the compound-of-interest, the sample comprises one or more components, preferably, 2 or more components, more preferably, 3 or more components. In general, a sample will comprise one compound-of-interest but can comprise multiple compounds-of-interest. Typically, a sample comprises less than about 1 g of the compound-of-interest, preferably, less than about 100 mg, more preferably, less than about 25 mg, even more preferably, less than about 1 mg, still more preferably less than about 100 micrograms, and optimally less than about 100 nanograms of the compound-of-interest. Preferably, the sample has a total volume of 100-250 ul.

A sample can be contained in any container or holder, or present on any substance or surface, or absorbed or adsorbed in any substance or surface. The only requirement is that the samples are isolated from one another; that is, located at separate sites. In one embodiment, samples are contained in sample wells in standard sample plates, for instance, in 24, 36, 48, or 96 well plates or more (or filter plates) of volume 250 ul commercially available, for example, from Millipore, Bedford, Mass.

In another embodiment, the samples can be contained in glass sample tubes. In this embodiment, the array consists of 96 individual glass tubes in a metal support plate. The tube is equipped with a plunger seal having a filter frit on the plunger top. The various components and the compound-of-interest are distributed to the tubes, and the tubes sealed. The sealing is accomplished by capping with a plug-type cap. Preferably, both the plunger and top cap are injection molded from thermoplastics, ideally chemically resistant thermoplastics such as PFA (although polyethylene and polypropylene are sufficient for less aggressive solvents). This tube design allows for both removal of solvent from tube as well as harvesting of solid-forms. Specifically, the plunger cap is pierced with a standard syringe needle and fluid is aspirated through the syringe tip to remove solvent form the tube. This can be accomplished by well-known methods. By having the frit barrier between the solvent and the syringe tip, the solid-form can be separated from the solvent. Once the solvent is removed, the plunger is then forced up the tube, effectively scraping any solid substance present on the walls, thereby collecting the solid-form on the frit. The plunger is fully extended at least to a level where the frit, and any collected solid-forms, are fully exposed above the tube. This allows the frit to be inserted into the under-side of a custom etched glass analysis plate. This analysis plate has 96 through-holes etched corresponding to the 96 individual frits. The top-side of the analysis plate has an optically-clear glass plate bonded onto it to both seal the plate as well as provide a window for analysis. The analysis plate assembly, which contains the plate itself plus the added frits with the solid-form, can be stored at room temperature, under an inert atmosphere if desired. The individual sample tube components are readily constructed from HPLC auto-sampler tube designs, for example, those of Waters Corp (Milford, Mass.). The automation mechanisms for capping, sealing, and sample tube manipulation are readily available to those skilled in the art of industrial automation.

4.3 Compound-of-Interest

The term “compound-of-interest” means the common component present in array samples where the array is designed to study its physical or chemical properties. Preferably, a compound-of-interests is a particular compound for which it is desired to identify solid-forms or solid-forms with enhanced properties. The compound-of-interest may also be a particular compound for which it is desired to find conditions or compositions that inhibit, prevent, or reverse solidification. Preferably, the compound-of-interest is present in every sample of the array, with the exception of negative controls. Examples of compounds-of-interest include, but are not limited to, pharmaceuticals, dietary supplements, alternative medicines, nutraceuticals, sensory compounds, agrochemicals, the active component of a consumer formulation, and the active component of an industrial formulation. Preferably, the compound-of-interest is a pharmaceutical. The compound-of-interest can be a known or novel compound. More preferably, the compound-of-interest is a known compound in commercial use.

4.3.1 Pharmaceutical

As used herein, the term “pharmaceutical” means any substance that has a therapeutic, disease preventive, diagnostic, or prophylactic effect when administered to an animal or a human. The term pharmaceutical includes prescription pharmaceuticals and over the counter pharmaceuticals. Pharmaceuticals suitable for use in the invention include all those known or to be developed. A pharmaceutical can be a large molecule (i.e., molecules having a molecular weight of greater than about 1000 g/mol), such as oligonucleotides, polynucleotides, oligonucleotide conjugates, polynucleotide conjugates, proteins, peptides, peptidomimetics, or polysaccharides or small molecules (i.e., molecules having a molecular weight of less than about 1000 g/mol), such as hormones, steroids, nucleotides, nucleosides, or aminoacids. Examples of suitable small molecule pharmaceuticals include, but are not limited to, cardiovascular pharmaceuticals, such as amlodipine, losartan, irbesartan, diltiazem, clopidogrel, digoxin, abciximab, furosemide, amiodarone, beraprost, tocopheryl; anti-infective components, such as amoxicillin, clavulanate, azithromycin, itraconazole, acyclovir, fluconazole, terbinafine, erytlromycin, and acetyl sulfisoxazole; psychotherapeutic components, such as sertaline, vanlafaxine, bupropion, olanzapine, buspirone, alprazolam, methylphenidate, fluvoxamine, and ergoloid; gastrointestinal products, such as lansoprazole, ranitidine, famotidine, ondansetron, granisetron, sulfasalazine, and infliximab; respiratory therapies, such as loratadine, fexofenadine, cetirizine, fluticasone, salmeterol, and budesonide; cholesterol reducers, such as atorvastatin calcium, lovastatin, bezafibrate, ciprofibrate, and gemfibrozil; cancer and cancer-related therapies, such as paclitaxel, carboplatin, tamoxifen, docetaxel, epirubicin, leuprolide, bicalutamide, goserelin implant, irinotecan, gemcitabine, and sargramostim; blood modifiers, such as epoetin alfa, enoxaparin sodium, and antihemophilic factor; antiarthritic components, such as celecoxib, nabumetone, misoprostol, and rofecoxib; AIDS and AIDS-related pharmaceuticals, such as lamivudine, indinavir, stavudine, and lamivudine; diabetes and diabetes-related therapies, such as metformin, troglitazone, and acarbose; biologicals, such as hepatitis B vaccine, and hepatitis A vaccine; hormones, such as estradiol, mycophenolate mofetil, and methylprednisolone; analgesics, such as tramadol hydrochloride, fentanyl, metamizole, ketoprofen, morphine, lysine acetylsalicylate, ketoralac tromethamine, loxoprofen, and ibuprofen; dermatological products, such as isotretinoin and clindamycin; anesthetics, such as propofol, midazolam, and lidocaine hydrochloride; migraine therapies, such as sumatriptan, zolmitriptan, and rizatriptan; sedatives and hypnotics, such as zolpidem, zolpidem, triazolam, and hycosine butylbromide; imaging components, such as iohexol, teclinetium, TC99M, sestamibi, iomeprol, gadodiamide, ioversol, and iopromide; and diagnostic and contrast components, such as alsactide, americium, betazole, histamine, mannitol, metyrapone, petagastrin, phentolamine, radioactive B₁₂, gadodiamide, gadopentetic acid, gadoteridol, and perflubron. Other pharmaceuticals for use in the invention include those listed in Table 1 below, which suffer from problems that could be mitigated by developing new administration formulations according to the arrays and methods of the invention.

TABLE I
Exemplary Pharmaceuticals

| Brand Name | Chemical | Properties |
|---|---|---|
| SANDIMMUNE | cyclosporin | Poor absorption in part |
| | | due to its low water |
| | | solubility. |
| TAXOL | paclitaxel | Poor absorption due to |
| | | its low water solubility. |
| VIAGRA | sildenafil citrate | Poor absorption due to |
| | | its low water solubility. |
| NORVIR | ritonavir | Can undergo a polymorphic |
| | | shift during shipping |
| | | and storage. |
| FULVICIN | griseofulvin | Poor absorption due to |
| | | its low water solubility. |
| FORTOVASE | saquinayir | Poor absorption due to |
| | | its low water solubility. |

Still other examples of suitable pharmaceuticals are listed in 2000 Med Ad News 19:56-60 and The Physicians Desk Reference, 53rd edition, 792-796, Medical Economics Company (1999), both of which are incorporated herein by reference.

Examples of suitable veterinary pharmaceuticals include, but are not limited to, vaccines, antibiotics, growth enhancing components, and dewormers. Other examples of suitable veterinary pharmaceuticals are listed in The Merck Veterinary Manual, 8th ed., Merck and Co., Inc., Rahway, N.J., 1998; (1997) The Encyclopedia of Chemical Technology, 24 Kirk-Othomer (4^{th} ed. at 826); and Veterinary Drugs in ECT 2nd ed., Vol 21, by A. L. Shore and R. J. Magee, American Cyanamid Co.

4.3.2 Dietary Supplement

As used herein, the term “dietary supplement” means a non-caloric or insignificant-caloric substance administered to an animal or a human to provide a nutritional benefit or a non-caloric or insignificant-caloric substance administered in a food to impart the food with an aesthetic, textural, stabilizing, or nutritional benefit. Dietary supplements include, but are not limited to, fat binders, such as caducean; fish oils; plant extracts, such as garlic and pepper extracts; vitamins and minerals; food additives, such as preservatives, acidulents, anticaking components, antifoaming components, antioxidants, bulking components, coloring components, curing components, dietary fibers, emulsifiers, enzymes, firming components, humectants, leavening components, lubricants, non-nutritive sweeteners, food-grade solvents, thickeners; fat substitutes, and flavor enhancers; and dietary aids, such as appetite suppressants. Examples of suitable dietary supplements are listed in (1994) The Encyclopedia of Chemical Technology, 11 Kirk-Othomer (4^{th} ed. at 805-833). Examples of suitable vitamins are listed in (1998) The Encyclopedia of Chemical Technology, 25 Kirk-Othomer (4^{th} ed. at 1) and Goodman & Gilman's: The Pharmacological Basis of Therapeutics, 9th Edition, eds. Joel G. Harman and Lee E. Limbird, McGraw-Hill, 1996 p. 1547, both of which are incorporated by reference herein. Examples of suitable minerals are listed in The Encyclopedia of Chemical Technology, 16 Kirk-Othomer (4^{th} ed. at 746) and “Mineral Nutrients” in ECT 3rd ed., Vol 15, pp. 570-603, by C. L. Rollinson and M. G. Enig, University of Maryland, both of which are incorporated herein by reference

4.3.3 Alternative Medicine

As used herein, the term “alternative medicine” means a substance, preferably a natural substance, such as a herb or an herb extract or concentrate, administered to a subject or a patient for the treatment of disease or for general health or well being, wherein the substance does not require approval by the FDA. Examples of suitable alternative medicines include, but are not limited to, ginkgo biloba, ginseng root, valerian root, oak bark, kava kava, echinacea, harpagophyti radix, others are listed in The Complete German Commission Monographs: Therapeutic Guide to Herbal Medicine, Mark Blumenthal et al. eds., Integrative Medicine Communications 1998, incorporated by reference herein.

4.3.4 Nutraceutical

As used herein the term “nutraceutical” means a food or food product having both caloric value and pharmaceutical or therapeutic properties. Example of nutraceuticals include garlic, pepper, brans and fibers, and health drinks Examples of suitable Nutraceuticals are listed in M. C. Linder, ed. Nutritional Biochemistry and Metabolism with Clinical Applications, Elsevier, New York, 1985; Pszczola et al., 1998 Food technology 52:30-37 and Shukla et al., 1992 Cereal Foods World 37:665-666.

4.3.5 Sensory Compound

As used herein, the term “sensory-material” means any chemical or substance, known or to be developed, that is used to provide an olfactory or taste effect in a human or an animal, preferably, a fragrance material, a flavor material, or a spice. A sensory-material also includes any chemical or substance used to mask an odor or taste. Examples of suitable fragrances materials include, but are not limited to, musk materials, such as civetone, ambrettolide, ethylene brassylate, musk xylene, Tonalide®, and Glaxolide®; amber materials, such as ambrox, ambreinolide and ambrinol; sandalwood materials, such as α-santalol, β-santalol, Sandalore®, and Bacdanol®; patchouli and woody materials, such as patchouli oil, patchouli alcohol, Timberol®and Polywood®; materials with floral odors, such as Givescone®, damascone, irones, linalool, Lilial®, Lilestralis®, and dihydrojasmonate. Other examples of suitable fragrance materials for use in the invention are listed in Perfumes: Art, Science, Technology, P. M. Muller ed. Elsevier, New York, 1991, incorporated herein by reference. Examples of suitable flavor materials include, but are not limited to, benzaldehyde, anethole, dimethyl sulfide, vanillin, methyl anthranilate, nootkatone, and cinnamyl acetate. Examples of suitable spices include but are not limited to allspice, tarrogon, clove, pepper, sage, thyme, and coriander. Other examples of suitable flavor materials and spices are listed in Flavor and Fragrance Materials-1989, Allured Publishing Corp. Wheaton, Ill., 1989; Bauer and Garbe Common Flavor and Fragrance Materials, VCH Verlagsgesellschaft, Weinheim, 1985; and (1994) The Encyclopedia of Chemical Technology, 11 Kirk-Othomer (4^{th} ed. at 1-61), all of which are incorporated by reference herein.

4.3.6 Agrochemical

As used herein, the term “agrochemical” means any substance known or to be developed that is used on the farm, yard, or in the house or living area to benefit gardens, crops, ornamental plants, shrubs, or vegetables or kill insects, plants, or fungi. Examples of suitable agrochemicals for use in the invention include pesticides, herbicides, fungicides, insect repellants, fertilizers, and growth enhancers. For a discussion of agrochemicals see The Agrochemicals Handbook (1987) 2nd Edition, Hartley and Kidd, editors: The Royal Society of Chemistry, Nottingham, England.

Pesticides include chemicals, compounds, and substances administered to kill vermin such as bugs, mice, and rats and to repel garden pests such as deer and woodchucks. Examples of suitable pesticides that can be used according to the invention include, but are not limited to, abamectin (acaricide), bifenthrin (acaricide), cyphenothrin (insecticide), imidacloprid (insecticide), and prallethrin (insectide). Other examples of suitable pesticides for use in the invention are listed in Crop Protection Chemicals Reference, 6th ed., Chemical and Pharmaceutical Press, John Wiley & Sons Inc., New York, 1990; (1996) The Encyclopedia of Chemical Technology, 18 Kirk-Othomer (4^{th} ed. at 311 -341); and Hayes et al., Handbook of Pesticide Toxicology, Academic Press, Inc., San Diego, Calif., 1990, all of which are incorporated by reference herein.

Herbicides include selective and non-selective chemicals, compounds, and substances administered to kill plants or inhibit plant growth. Examples of suitable herbicides include, but are not limited to, photosystem I inhibitors, such as actifluorfen; photosystem II inhibitors, such as atrazine; bleaching herbicides, such as fluridone and difunon; chlorophyll biosynthesis inhibitors, such as DTP, clethodim, sethoxydim, methyl haloxyfop, tralkoxydim, and alacholor; inducers of damage to antioxidative system, such as paraquat; amino-acid and nucleotide biosynthesis inhibitors, such as phaseolotoxin and imazapyr; cell division inhibitors, such as pronamide; and plant growth regulator synthesis and function inhibitors, such as dicamba, chloramben, dichlofop, and ancymidol. Other examples of suitable herbicides are listed in Herbicide Handbook, 6th ed., Weed Science Society of America, Champaign, II 1989; (1995) The Encyclopedia of Chemical Technology, 13 Kirk-Othomer (4^{th} ed. at 73-136); and Duke, Handbook of Biologically Active Phytochemicals and Their Activities, CRC Press, Boca Raton, Fla., 1992, all of which are incorporated herein by reference.

Fungicides include chemicals, compounds, and substances administered to plants and crops that selectively or non-selectively kill fungi. For use in the invention, a fungicide can be systemic or non-systemic. Examples of suitable non-systemic fungicides include, but are not limited to, thiocarbamate and thiurame derivatives, such as ferbam, ziram, thiram, and nabam; imides, such as captan, folpet, captafol, and dichlofluanid; aromatic hydrocarbons, such as quintozene, dinocap, and chloroneb; dicarboximides, such as vinclozolin, chlozolinate, and iprodione. Example of systemic fungicides include, but are not limited to, mitochondiral respiration inhibitors, such as carboxin, oxycarboxin, flutolanil, fenfuram, mepronil, and methfuroxam; microtubulin polymerization inhibitors, such as thiabendazole, fuberidazole, carbendazim, and benomyl; inhibitors of sterol biosynthesis, such as triforine, fenarimol, nuarimol, imazalil, triadimefon, propiconazole, flusilazole, dodemorph, tridemorph, and fenpropidin; and RNA biosynthesis inhibitors, such as ethirimol and dimethirimol; phopholipic biosynthesis inhibitors, such as ediphenphos and iprobenphos. Other examples of suitable fungicides are listed in Torgeson, ed., Fungicides: An Advanced Treatise, Vols. 1 and 2, Academic Press, Inc., New York, 1967 and (1994) The Encyclopedia of Chemical Technology, 12 Kirk-Othomer (4^{th} ed. at 73-227), all of which are incorporated herein by reference.

4.3.7 Consumer and Industrial Formulations

The arrays and methods of the invention can be used to identify new solid-forms of the components of consumer and industrial formulations. As used herein, a “consumer formulation” means a formulation for consumer use, not intended to be absorbed or ingested into the body of a human or animal, comprising an active component. Preferably, it is the active component that is investigated as the compound-of-interest in the arrays and methods of the invention. Consumer formulations include, but are not limited to, cosmetics, such as lotions, facial makeup; antiperspirants and deodorants, shaving products, and nail care products; hair products, such as and shampoos, colorants, conditioners; hand and body soaps; paints; lubricants; adhesives; and detergents and cleaners.

As used herein an “industrial formulation” means a formulation for industrial use, not intended to be absorbed or ingested into the body of a human or animal, comprising an active component. Preferably, it is the active component of industrial formulation that is investigated as the compound-of-interest in the arrays and methods of the invention. Industrial formulations include, but are not limited to, polymers; rubbers; plastics; industrial chemicals, such as solvents, bleaching agents, inks, dyes, fire retardants, antifreezes and formulations for deicing roads, cars, trucks, jets, and airplanes; industrial lubricants; industrial adhesives: construction materials, such as cements.

One of skill in the art will readily be able to choose active components and inactive components used in consumer and industrial formulations and set up arrays according to the invention. Such active components and inactive components are well known in the literature and the following references are provided merely by way of example. Active components and inactive components for use in cosmetic formulations are listed in (1993) The Encyclopedia of Chemical Technology, 7 Kirk-Othomer (4^{th} ed. at 572-619); M. G. de Navarre, The Chemistry and Manufacture of Cosmetics, D. Van Nostrand Company, Inc., New York, 1941; CTFA International Cosmetic Ingredient Dictionary and Handbook, 8th Ed., CTFA, Washington, D.C., 2000; and A. Nowak, Cosmetic Preparations, Micelle Press, London, 1991. All of which are incorporated by reference herein. Active components and inactive components for use in hair care products are listed in (1994) The Encyclopedia of Chemical Technology 12 Kirk-Othomer (4^{th} ed. at 881-890) and Shampoos and Hair Preparations in ECT 1st ed., Vol. 12, pp. 221-243 by F. E. Wall, both of which are incorporated by reference herein. Active components and inactive components for use in hand and body soaps are listed in (1997) The Encyclopedia of Chemical Technology, 22 Kirk-Othomer (4^{th} ed. at 297-396), incorporated by reference herein. Active components and inactive components for use in paints are listed in (1996) The Encyclopedia of Chemical Technology, 17 Kirk-Othomer (4^{th} ed. at 1049-1069) and “Paint” in ECT 1st ed., Vol. 9. pp. 770-803, by H. E. Hillman, Eagle Paint and Varnish Corp, both of which are incorporated by reference herein. Active components and inactive components for use in consumer and industrial lubricants are listed in (1995) The Encyclopedia of Chemical Technology, 15 Kirk-Othomer (4^{th} ed. at 463-517); D. D. Fuller, Theory and practice of Lubrication for Engineers, 2nd ed., John Wiley & Sons, Inc., 1984; and A. Raimondi and A. Z. Szeri, in E. R. Booser, eds., Handbook of Lubrication, Vol. 2, CRC Press Inc., Boca Raton, Fla., 1983, all of which are incorporated by reference herein. Active components and inactive components for use in consumer and industrial adhesives are listed in (1991) The Encyclopedia of Chemical Technology, 1 Kirk-Othomer (4^{th} ed. at 445-465) and I. M. Skeist. ed. Handbook of Adhesives, 3rd ed. Van Nostrand-Reinhold, New York, 1990, both of which are incorporated herein by reference. Active components and inactive components for use in polymers are listed in (1996) The Encyclopedia of Chemical Technology, 19 Kirk-Othomer (4^{th} ed. at 881-904), incorporated herein by reference. Active components and inactive components for use in rubbers are listed in (1997) The Encyclopedia of Chemical Technology, 21 Kirk-Othomer (4^{th} ed. at 460-591), incorporated herein by reference. Active components and inactive components for use in plastics are listed in (1996) The Encyclopedia of Chemical Technology, 19 Kirk-Othomer (4^{th} ed. at 290-316), incorporated herein by reference. Active components and inactive components for use with industrial chemicals are listed in Ash et al., Handbook of Industrial Chemical Additives, VCH Publishers, New York 1991, incorporated herein by reference. Active components and inactive components for use in bleaching components are listed in (1992) The Encyclopedia of Chemical Technology, 4 Kirk-Othomer (4^{th} ed. at 271-311), incorporated herein by reference. Active components and inactive components for use inks are listed in (1995) The Encyclopedia of Chemical Technology, 14 Kirk-Othomer (4^{th} ed. at 482-503), incorporated herein by reference. Active components and inactive components for use in dyes are listed in (1993) The Encyclopedia of Chemical Technology, 8 Kirk-Othomer (4^{th} ed. at 533-860), incorporated herein by reference. Active components and inactive components for use in fire retardants are listed in (1993) The Encyclopedia of Chemical Technology, 10 Kirk-Othomer (4^{th} ed. at 930-1022), incorporated herein by reference. Active components and inactive components for use in antifreezes and deicers are listed in (1992) The Encyclopedia of Chemical Technology, 3 Kirk-Othomer (4^{th} ed. at 347-367), incorporated herein by reference. Active components and inactive components for use in cement are listed in (1993) The Encyclopedia of Chemical Technology, 5 Kirk-Othomer (4^{th} ed. at 564), incorporated herein by reference.

4.4 Component

As used herein, the term “component” means any substance that is combined, mixed, or processed with the compound-of-interest to form a sample or impurities, for example, trace impurities left behind after synthesis or manufacture of the compound-of-interest. The term component also encompasses the compound-of-interest itself. The term component also includes any solvents in the sample. A single substance can exist in one or more physical states having different properties thereby classified herein as different components. For instance, the amorphous and crystalline forms of an identical compound are classified as different components. Components can be large molecules (i.e., molecules having a molecular weight of greater than about 1000 g/mol), such as large-molecule pharmaceuticals, oligonucleotides, polynucleotides, oligonucleotide conjugates, polynucleotide conjugates, proteins, peptides, peptidomimetics, or polysaccharides or small molecules (i.e., molecules having a molecular weight of less than about 1000 g/mol) such as small-molecule pharmaceuticals, hormones, nucleotides, nucleosides, steroids, or aminoacids. Components can also be chiral or optically-active substances or compounds, such as optically-active solvents, optically-active reagents, or optically-active catalysts. Preferably, components promote or inhibit or otherwise effect precipitation, formation, crystallization, or nucleation of solid-forms, preferably, solid-forms of the compound-of-interest. Thus, a component can be a substance whose intended effect in an array sample is to induce, inhibit, prevent, or reverse formation of solid-forms of the compound-of-interest. Examples of components include, but are not limited to, excipients; solvents; salts; acids; bases; gases; small molecules, such as hormones, steroids, nucleotides, nucleosides, and aminoacids; large molecules, such as oligonucleotides, polynucleotides, oligonucleotide and polynucleotide conjugates, proteins, peptides, peptidomimetics, and polysaccharides; pharmaceuticals; dietary supplements; alternative medicines; nutraceuticals; sensory compounds; agrochemicals; the active component of a consumer formulation; and the active component of an industrial formulation; crystallization additives, such as additives that promote and/or control nucleation, additives that affect crystal habit, and additives that affect polymorphic form; additives that affect particle or crystal size; additives that structurally stabilize crystalline or amorphous solid-forms; additives that dissolve solid-forms; additives that inhibit crystallization or solid formation; optically-active solvents; optically-active reagents; optically-active catalysts; and even packaging or processing reagents.

4.4.1 Excipient

The term “excipient” as used herein means the substances used to formulate actives into pharmaceutical formulations. Preferably, an excipient does not lower or interfere with the primary therapeutic effect of the active, more preferably, an excipient is therapeutically inert. The term “excipient” encompasses carriers, solvents, diluents, vehicles, stabilizers, and binders. Excipients can also be those substances present in a pharmaceutical formulation as an indirect result of the manufacturing process. Preferably, excipients are approved for or considered to be safe for human and animal administration, i.e., GRAS substances (generally regarded as safe). GRAS substances are listed by the Food and Drug administration in the Code of Federal Regulations (CFR) at 21 CFR 182 and 21 CFR 184, incorporated herein by reference.

Bioactive substances (e.g., pharmaceuticals) can be formulated as tablets, powders, particles, solutions, suspensions, patches, capsules, with coatings, excipients, or packaging that farther affects the delivery properties, the biological properties, and stability during storage, as well as formation of solid-forms. An excipient may also be used in preparing the sample, for example, by coating the surface of the sample tubes or sample wells in which the component-of-interest is being crystallized, or by being present in the crystallizing solution at different concentrations. For example, variations in surfactant composition can also be used to create diversity in crystalline form. Maximum variation in surfactant composition can be achieved, for example, in the case of a protein surfactant, by varying the protein composition using techniques currently used to create large libraries of protein variants. These techniques include mutating systematically randomly the DNA encoding the protein's amino acid sequence. Examples of suitable excipients include, but are not limited to, acidulents, such as lactic acid, hydrochloric acid, and tartaric acid; solubilizing components, such as non-ionic, cationic, and anionic surfactants; absorbents, such as bentonite, cellulose, and kaolin; alkalizing components, such as diethanolamine, potassium citrate, and sodium bicarbonate; anticaking components, such as calcium phosphate tribasic, magnesium trisilicate, and talc; antimicrobial components, such as behzoic acid, sorbic acid, benzyl alcohol, benzethonium chloride, bronopol, alkyl parabens, cetrimide, phenol, phenylmercuric acetate, thimerosol, and phenoxyethanol; antioxidants, such as ascorbic acid, alpha tocopherol, propyl gallate, and sodium metabisulfite; binders, such as acacia, alginic acid, carboxymethyl cellulose, hydroxyethyl cellulose; dextrin, gelatin, guar gum, magnesium aluminum silicate, maltodextrin. povidone, starch, vegetable oil, and zein; buffering components, such as sodium phosphate, malic acid, and potassium citrate; chelating components, such as EDTA, malic acid, and maltol; coating components such as adjunct sugar, cetyl alcohol, polyvinyl alcohol, carnauba wax, lactose maltitol, titanium dioxide; controlled release vehicles, such as microcrystalline wax, white wax, and yellow wax; desiccants, such as calcium sulfate; detergents, such as sodium lauryl sulfate; diluents, such as calcium phosphate, sorbitol, starch, talc, lactitol, polymethacrylates, sodium chloride, and glyceryl palmitostearate; disintegrants, such as collodial silicon dioxide, croscarmellose sodium, magnesium aluminum silicate, potassium polacrilin, and sodium starch glycolate; dispersing components, such as poloxamer 386, and polyoxyethylene fatty esters (polysorbates); emollients, such as cetearyl alcohol, lanolin, mineral oil, petrolatum, cholesterol, isopropyl myristate, and lecithin; emulsifying components, such as anionic emulsifying wax, monoethanolamine, and medium chain triglycerides; flavoring components, such as ethyl maltol, ethyl vanillin, fumaric acid, malic acid, maltol, and menthol; humectants, such as glycerin, propylene glycol, sorbitol, and triacetin; lubricants, such as calcium stearate, canola oil, glyceryl palmitosterate, magnesium oxide, poloxymer, sodium benzoate, stearic acid, and zinc stearate; solvents, such as alcohols, benzyl phenylformate, vegetable oils, diethyl phthalate, ethyl oleate, glycerol, glycofurol, for indigo carmine, polyethylene glycol, for sunset yellow, for tartazine, triacetin; stabilizing components, such as cyclodextrins, albumin, xanthan gum; and tonicity components, such as glycerol, dextrose, potassium chloride, and sodium chloride; and mixture thereof. Other examples of suitable excipients, such as binders and fillers are listed in Remington's Pharmaceutical Sciences, 18th Edition, ed. Alfonso Gennaro, Mack Publishing Co. Easton, Pa., 1995 and Handbook of Pharmaceutical Excipients, 3rd Edition, ed. Arthur H. Kibbe, American Pharmaceutical Association, Washington D.C. 2000, both of which are incorporated herein by reference.

4.4.2 Solvents

In general, arrays of the invention will contain a solvent as one on the components. Solvents may influence and direct the formation of solid-forms through polarity, viscosity, boiling point, volatility, charge distribution, and molecular shape. The solvent identity and concentration is one way to control saturation. Indeed, one can crystallize under isothermal conditions by simply adding a nonsolvent to an initially subsaturated solution. One can start with an array of a solution of the compound-of-interest in which varying amounts of nonsolvent are added to each of the individual elements of the array. The solubility of the compound is exceeded when some critical amount of nonsolvent is added. Further addition of the nonsolvent increases the supersaturation of the solution and, therefore, the growth rate of the crystals that are grown. Mixed solvents also add the flexibility of changing the thermodynamic activity of one of the solvents independent of temperature. Thus, one can select which hydrate or solvate is produced at a given temperature simply by carrying out crystallization over a range of solvent compositions. For example, crystallization from a methanol-water solution that is very rich in methanol will favor solid-form hydrates with fewer waters incorporated in the solid (ex. dihydrate vs. hemihydrate) while a water rich solution will favor hydrates with more waters incorporated into the solid. The precise boundaries for producing the respective hydrates are found by examining the elements of the array when concentration of the solvent component is the variable.

Specific applications may create additional requirements. For example, in the case of pharmaceuticals, solvents are selected based on their biocompatibility as well as the solubility of the pharmaceutical to be crystallized, and in some cases, the excipients. For example, the ease with which the agent is dissolved in the solvent and the lack of detrimental effects of the solvent on the agent are factors to consider in selecting the solvent. Aqueous solvents can be used to make matrices formed of water soluble polymers. Organic solvents will typically be used to dissolve hydrophobic and some hydrophilic polymers. Preferred organic solvents are volatile or have a relatively low boiling point or can be removed under vacuum and that are acceptable for administration to humans in trace amounts, such as methylene chloride. Other solvents, such as ethyl acetate, ethanol, methanol, dimethyl formamide, acetone, acetonitrile, tetrahydrofuran, acetic acid, dimethyl sulfoxide, and chloroform, and mixture thereof, also can be used. Preferred solvents are those rated as class 3 residual solvents by the Food and Drug Administration, as published in the Federal Register vol. 62, number 85, pp. 24301-24309 (May 1997). Solvents for pharmaceuticals that are administered parenterally or as a solution or suspension will more typically be distilled water, buffered saline, Lactated Ringer's or some other pharmaceutically acceptable carrier.

4.4.3 Components Capable of Forming Salts: Acidic and Basic Components

The term “components” includes acidic substances and basic substances. Such substances can react to form a salt with the compound-of-interest or other components present in a sample. When a salt of the compound-of-interest is desired, salt forming components will generally be used in stoichiometric quantities. Components that are basic in nature are capable of forming a wide variety of salts with various inorganic and organic acids. For example, suitable acids are those that form the following salts with basic compounds: chloride, bromide, iodide, acetate, salicylate, benzenesulfonate, benzoate, bicarbonate, bitartrate, calcium edetate, camsylate, carbonate, citrate, edetate, edisylate, estolate, esylate, fumarate, gluceptate, gluconate, glutatate, glycollylarsanilate. hexylresorcinate, hydrabamine, hydroxynaphthoate, isethionate, lactate, lactobionate, malate, maleate, mandelate, mesylate, methylsulfate, muscate, napsylate, nitrate, panthothenate, phosphate/diphosphate, polygalacturonate, salicylate, stearate, succinate, sulfate, tannate, tartrate, teoclate, triethiodide, and pamoate (i.e., 1,1′-methylene-bis-(2-hydroxy-3-naphthoate)). Components that include an amino moiety also can form pharmaceutically-acceptable salts with various amino acids, in addition to the acids mentioned above.

Compounds-of-interest that are acidic in nature are capable of forming base salts with various cations. Examples of such salts include alkali metal or alkaline earth metal salts and, particularly, calcium, magnesium, sodium, lithium, zinc, potassium, and iron salts, as well as salts of basic organic compounds, such as amines, for example N-methylglucamine and TRIS (tris-hydroxymethyl aminomethane).

4.4.4 Crystallization Additives

Other substances may also be added to the crystallization reactions whose presence will influence the generation of a crystalline form. These crystallization additives can be either reaction by products, or related molecules, or randomly screened compounds (such as those present in small molecule libraries). They can be used to either promote or control nucleation, to direct the growth or growth rate of a specific crystal or set of crystals, and any other parameter that affects crystallization. The influence of crystallization additives may depend on their relative concentrations and thus the invention provides methods to assess a range of crystallization additives and concentrations. Examples of crystallization additives include, but are not limited to, additives that promote and/or control nucleation, additives that affect crystal habit, and additives that affect polymorphic form.

4.4.4.1 Additives that Promote and/or Control Nucleation

The presence of surfactant-like molecules in the crystallization vessel may influence the crystal nucleation and selectively drive the growth of distinct polymorphic forms. Thus, surfactant-like molecules can be introduced into the crystallization vessel either by pre-treating the microtiter dishes or by direct addition to the crystallization medium. Surfactant molecules can be either specifically selected or randomly screened for their influence in directing crystallization. In addition, the effect of the surfactant molecule is dependent on its concentration in the crystallization vessel and thus the concentration of the surfactant molecules should be carefully controlled.

In some cases, direct seeding of crystallization reactions will result in an increased diversity of crystal forms being produced. In one embodiment, particles are added to the crystallization reactions. In another, nanometer-sized crystals (nanoparticles) are added to the crystallization reactions. In still another embodiment, other substances can be used including solid phase GRAS compounds or alternatively, small molecule libraries (in solid phase). These particles can be either nanometer sized or larger.

In addition to the compound to be screened, solvents, seeds, and nucleating agents, other substances can be added to the crystallization reactions whose presence will influence the generation of a particular solid phase form. These crystallization additives can be either reaction by products, or related molecules, or randomly screened compounds (such as those present in small molecule libraries). The influence of crystallization additives to direct the growth of a specific crystal or set of crystals may also depend on their relative concentrations and thus it is anticipated that a range of crystallization additive concentrations will need to be assessed.

4.4.4.2 Additives that Affect Crystal Habit

Small amounts of soluble species can also dramatically affect the habit or size of the crystals that are grown without having a marked influence on the pharmaceutical's solubility. The influence of impurities on crystal habit or size modification has been known for many years. The crystallization additives often are similar in form to the host molecule or pharmaceutical and have a stereo-chemical relationship to specific crystal faces. That is, the ability to absorb on a given crystal face can be restricted by the stereo-chemical structure of the crystallization additive and the symmetry of the crystal face. Selective absorption on various faces of the crystal can affect the growth rate of that face. Thus, the habit of the crystal will change.

4.4.4.3 Additives Affect Polymorphic Form

As discussed above, the same compound can crystallize as more than one distinct crystalline species (i.e., having a different internal structure). This phenomena is known as polymorphism, and the distinct species are known as polymorphs. Discovery of additives that direct formation of one polymorph over another or promote conversion of a less stable polymorph into the more stable form are of considerable importance, for example, in the pharmaceutical industry, where certain polymorphs of a given pharmaceutical are more therapeutically beneficial than other forms. Seed crystals of a given polymorph can be used as additives in subsequent crystallizations to direct polymorph formation.

4.4.5 Additives that Affect Particle or Crystal Size

Particulate matter, produced by precipitation of amorphous particles or crystallization, has a distribution of sizes that varies in a definite way over throughout the size range. Control of particle or crystal size is very important in pharmaceutical compounds. The smaller the crystal size, the higher the surface-to-volume ratio. In general, finding additives that affect particle or crystal size is a mix and try process with few general rules available in the literature. Many substances can affect particle or crystal size, for example solvents, excipients, solvents, nucleation promoters, such as surfactants, particulate matter, the physical state of crystal seeds, and even trace amounts of impurities.

4.4.6 Additives that Stabilize the Structure of Crystalline or Amorphous Solid-Forms

Molecules can crystallize in more than one polymorphic form. A less thermodynamically stable polymorph can spontaneously convert to the more stable form if the phase transition barrier is overcome. This is undesirable, for example, when the less thermodynamically stable polymorphic form of a pharmaceutical is more pharmacologically advantageous than the more stable form. Thus, inhibitors of polymorphic shift are much needed, especially for stabilization of metastable polymorphic pharmaceuticals. Polymorphic shift inhibitors can act by a variety of mechanisms including stabilizing the crystal surface. In general, at conditions close to equilibrium, only the thermodynamically stable polymorph will be formed. Those substances that inhibit crystallization of the more stable polymorphic form under these equilibrium conditions are potential stabilizers for a less stable, but possibly more desirable polymorphic form. A properly designed inhibitor should preferentially interact with pre-critical nuclei of the stable crystalline phase but not with the less stable phase (desired polymorph). Strong inhibition can result in preferential kinetic crystallization of the less stable polymorph.

4.4.7 Additives that Inhibit Crystallization or Precipitation and/or Dissolve Solids or Prevent Solid Formation

Crystallization inhibitors can be used for a variety of purposes including morphological engineering, etching, reduction in crystal symmetry, and elucidating the effect of components on crystal growth (see e.g., Weissbuch et al., 1995 Acta Cryst. B51:115-148). Tailor made crystal growth inhibitors that interact with specific crystal faces have been reported, see e.g., Addadi et al., (1985) Agnew. Chem. Int. Ed. Engl. 24:466-485 and Weissbuch et al., (1991) Science 253:637-645. Crystallization inhibitors have many important applications, for example, they are extremely useful in transdermal delivery systems. Such systems generally comprise a liquid phase reservoir containing the active component. But if the active component crystalizes, it is no longer available for transdermal delivery. Of course, the same goes for creams, gels, suspensions, and syrups designed for topical application.

Crystal growth inhibitors can affect the crystal habit, for example, when crystal growth is inhibited in a direction perpendicular to a given crystal face, the area of this face is expected to increase relative to the areas to the areas of other faces on the same crystal. Differences in the relative surface areas of the various faces can therefore be directly correlated to the inhibition in different growth directions.

Echants can promote dissolution of crystals thereby inducing the formation of etch pits on crystal faces or completely dissolving of the crystal. Weissbuch et al., 1995 Acta Cryst. B51:115-148. Dissolution or etching of a crystal occurs when the crystal is immersed in an unsaturated solution. Etchants refers to additives that effect the rate of this process. In Some cases, they actually interact with the crystal surface and can increase the presence of steps or ledges where the activation energy of dissolution is lower.

4.5 Processing Parameters

As used herein, the term “processing parameters” means the physical or chemical conditions under which a sample is subjected and the time during which the sample is subjected to such conditions. Processing parameters include, but are not limited to, adjusting the temperature; adjusting the time; adjusting the pH; adjusting the amount or the concentration of the compound-of-interest; adjusting the amount or the concentration of a component; component identity (adding one or more additional components); adjusting the solvent removal rate; introducing of a nucleation event; introducing of a precipitation event; controlling evaporation of the solvent (e.g., adjusting a value of pressure or adjusting the evaporative surface area); and adjusting the solvent composition.

Sub-arrays or even individual samples within an array can be subjected to processing parameters that are different from the processing parameters to which other sub-arrays or samples, within the same array, are subjected. Processing parameters will differ between sub-arrays or samples when they are intentionally varied to induce a measurable change in the sample's properties. Thus, according to the invention, minor variations, such as those introduced by slight adjustment errors, are not considered intentionally varied.

4.6 Property

As used herein, the term “property” means a structural, physical, pharmacological, or chemical characteristic of a sample, preferably, a structural, physical, pharmacological, or chemical characteristics of a compound-of-interest. Structural properties include, but are not limited to, whether the compound-of-interest is crystalline or amorphous, and if crystalline, the polymorphic form and a description of the crystal habit. Structural properties also include the composition, such as whether the solid-form is a hydrate, solvate, or a salt.

Preferred properties are those that relate to the efficacy, safety, stability, or utility of the compound-of-interest. For example, regarding pharmaceutical, dietary supplement, alternative medicine, and nutraceutical compounds and substances, properties include physical properties, such as stability, solubility, dissolution, permeability, and partitioning; mechanical properties, such as compressibility, compactability, and flow characteristics; the formulation's sensory properties, such as color, taste, and smell; and properties that affect the utility, such as absorption, bioavailability, toxicity, metabolic profile, and potency. Other properties include those which affect the compound-of-interest's behavior and ease of processing in a crystallizer or a formulating machine. For a discussion of industrial crystallizers and properties thereof see (1993) The Encyclopedia of Chemical Technology, 7 Kirk-Othomer (4^{th} ed. pp. 720-729). Such processing properties are closely related to the solid-form's mechanical properties and its physical state, especially degree of agglomeration. Concerning pharmaceuticals, dietary supplements, alternative medicines, and nutraceuticals, optimizing physical and utility properties of their solid-forms can result in a lowered required dose for the same therapeutic effect. Thus, there are potentially fewer side effects that can improve patient compliance.

Another important structural property is the surface-to-volume ratio and the degree of agglomeration of the particles. Surface-to-volume ratio decreases with the degree of agglomeration. It is well known that a high surface-to-volume ratio improves the solubility rate. Small-size particles have high surface-to-volume ratio. The surface-to-volume ratio is also influenced by the crystal habit, for example, the surface-to-volume ratio increases from spherical shape to needle shape to dendritic shape. Porosity also affects the surface-to-volume ratio, for example, solid-forms having channels or pores (e.g., inclusions, such as hydrates and solvates) have a high surface-to-volume ratio.

Still another structural property is particle size and particle-size distribution. For example, depending on concentrations, the presence of inhibitors or impurities, and other conditions, particles can form from solution in different sizes and size distributions. Particulate matter, produced by precipitation or crystallization, has a distribution of sizes that varies in a definite way throughout the size range. Particle- and crystal-size distribution is generally expressed as a population distribution relating to the number of particles at each size. In pharmaceuticals, particle and crystal size distribution have very important clinical aspects, such as bioavailability. Thus, compounds or compositions that promote small crystal size can be of clinical importance.

Physical properties include, but are not limited to, physical stability, melting point, solubility, strength, hardness, compressibility, and compactability. Physical stability refers to a compound's or composition's ability to maintain its physical form, for example maintaining particle size; maintaining crystal or amorphous form; maintaining complexed form, such as hydrates and solvates; resistance to absorption of ambient moisture; and maintaining of mechanical properties, such as compressibility and flow characteristics. Methods for measuring physical stability include spectroscopy, sieving or testing, microscopy, sedimentation, stream scanning, and light scattering. Polymorphic changes, for example, are usually detected by differential scanning calorimetry or quantitative infrared analysis. For a discussion of the theory and methods of measuring physical stability see Fiese et al., in The Theory and Practice of Industrial Pharmacy, 3rd ed., Lachman L.; Lieberman, H. A.; and Kanig, J. L. Eds., Lea and Febiger, Philadelphia, 1986 pp. 193-194 and Remington's Pharmaceutical Sciences, 18th Edition, ed. Alfonso Gennaro, Mack Publishing Co. Easton, Pa., 1995, pp. 1448-1451, both of which are incorporated herein by reference.

Chemical properties include, but are not limited to chemical stability, such as susceptibility to oxidation and reactivity with other compounds, such as acids, bases, or chelating agents. Chemical stability refers to resistance to chemical reactions induced, for example, by heat, ultraviolet radiation, moisture, chemical reactions between components, or oxygen. Well known methods for measuring chemical stability include mass spectroscopy, UV-VIS spectroscopy, HPLC, gas chromatography, and liquid chromatography-mass spectroscopy (LC-MS). For a discussion of the theory and methods of measuring chemical stability see Xu et al., Stability-Indicating HPLC Methods for Drug Analysis American Pharmaceutical Association, Washington D.C. 1999 and Remington's Pharmaceutical Sciences, 18th Edition, ed. Alfonso Gennaro, Mack Publishing Co. Easton, Pa., 1995, pp. 1458-1460, both of which are incorporated herein by reference.

4.7 Solid-Form

As used herein, the term “solid-form” means a form of a solid substance, element, or chemical compound that is defined and differentiated from other solid-forms according to its physical state and properties.

4.8 Physical State

According to the invention described herein, the “physical state” of a component or a compound-of-interest is initially defined by whether the component is a liquid or a solid. If the component is a solid, the physical state is further defined by the particle or crystal size and particle-size distribution.

Physical state also includes agglomeration and degree of agglomeration. Often processing solid-forms, such as crystals, in an industrial crystallizer requires that the solid-form be removed as small particles or single crystals. Thus, the ease of handling and many of the solid-form's properties can be affected deleteriously by agglomeration. For example, in addition to making the compound difficult to process, purity can be diminished when agglomeration occurs. Agglomeration can be accounted for by identifying relevant processing variables, such as crystals coming together and bonding through overgrowth of the contact area.

Physical state can further be defined by purity or the composition of the solid-form. Thus physical state includes whether a particular substance forms co-crystals with one or more other substances or compounds. Composition also includes whether the solid-form is in the form of a salt or contains a guest molecule or is impure. Mechanisms by which guest compounds or impurities can be incorporated in solid-forms include surface absorption and entrapment in cracks and crevices, especially in agglomerates and crystals.

Physical state includes whether the substance is crystalline or amorphous. If the substance is crystalline, the physical state is further divided into: (1) whether the crystal matrix includes a co-adduct; (2) morphology, i.e., crystal habit; and (3) internal structure (polymorphism). In a co-adduct, the crystal matrix can include either a stoichiometric or non-stoichiometric amount of the adduct, for example, a crystallization solvent or water, i.e., a solvate or a hydrate.

Non-stoichiometric solvates and hydrates include inclusions or clathrates, that is, where a solvent or water is trapped at random intervals within the crystal matrix, for example, in channels.

A stoichiometric solvate or hydrate is where a crystal matrix includes a solvent or water at specific sites in a specific ratio. That is, the solvent or water molecule is part of the crystal matrix in a defined arrangement. Additionally, the physical state of a crystal matrix can change by removing a co-adduct, originally present in the crystal matrix. For example, if a solvent or water is removed from a solvate or a hydrate, a hole is formed within the crystal matrix, thereby forming a new physical state. Such physical states are referred to herein as dehydrated hydrates or desolvated solvates.

The crystal habit is the description of the outer appearance of an individual crystal, for example, a crystal may have a cubic, tetragonal, orthorhombic, monoclinic, triclinic, rhomboidal, or hexagonal shape.

The internal structure of a crystal refers to the crystalline form or polymorphism. A given compound may exist as different polymorphs, that is, distinct crystalline species. In general, different polymorphs of a given compound are as different in structure and properties as the crystals of two different compounds. Solubility, melting point, density, hardness, crystal shape, optical and electrical properties, vapor pressure, and stability, etc. all vary with the polymorphic form.

4.9 Diastereomeric Derivatives of the Compound-of-Interest

A diastereomeric derivative of the compound-of-interest means the reaction product, salt, or complex resulting from treatment of a compound-of-interest having one or more chiral centers with a substrate compound having at least one chiral center. Preferably the substrate compound is optically enriched, preferably, having an enantiomeric excess of at least about 90%, more preferably, at least about 95%. A diastereomeric derivative can be in the form of an ionic salt, a covalent compound, a charge-transfer complex, or an inclusion compound (host-guest relationship). Preferably, the substrate compound can be readily cleaved to reform the compound-of-interest.

4.10 Stereoisomerically Enriched

The compound-of-interest can contain one or more chiral centers and/or double bonds and, therefore, exist as stereoisomers, such as double-bond isomers (i.e., geometric isomers), enantiomers, or diastereomers. As used herein, the term “stereoisomerically enriched” means that one stereoisomer is present in an amount greater than its statistically calculated amount. For example, and a compound with 1 or more chiral centers is statistically calculated to comprise two enantiomers in an amount of 50% each. Thus a compound is enantiomerically-enriched (optically active) when the compound has an enantiomeric excess of greater than about 1% ee, preferably, greater than about 25% ee, more preferably, greater than about 75% ee, even more preferably, greater than about 90% ee. As used herein, a racemic mixture means 50% of one en antiomer and 50% of is corresponding enantiomer. A compound with two or more chiral centers comprises a mixture of 2ⁿ diastereomers, where n is the number of chiral centers. A compound is considered diastereomerically enriched when one of die diastereomers is present in an amount greater than ½ⁿ% of all the diastereomers. Thus a compound containing 3 chiral centers comprises 8 diastereomers and if one of the diastereomers is present in an amount of greater than 12.5% (e.g., 13%), the compound is considered diastereomerically enriched. In another example, if a racemic mixture is treated with an optically pure compound to form a pair of diastereomers, each diastereomer is calculated to be present in an amount of 50%. If such a diastereomeric pair is resolved such that one diastereomer is present in greater than 50%, the compound is considered diastereomerically enriched.

4.11 Conglomerate

As used herein, a “conglomerate” means a compound that under certain conditions, crystallizes to yield optically-pure, discrete crystals or crystal clusters of both enantiomers. Preferably, such discrete crystals can be mechanically separated to yield the compound in enantiomerically-enriched form.

### 5. BRIEF DESCRIPTION OF THE FIGURES

FIG. 1 is a schematic of the high-throughput process for preparing arrays of solid-forms of a compound-of-interest and analyzing the individual samples.

FIG. 2A is a more detailed schematic of a system for high-throughput combinatorial mixing of components, incubation and dynamic analysis of samples, and in-depth characterization of lead candidates.

FIG. 2B is a schematic of the details of the sample preparation module depicted in FIG. 2A.

FIG. 2C is a schematic of the details of the incubation and dynamic scanning and in-depth characterization modules shown in FIG. 2A.

FIGS. 3A-3C are schematics of processes to generate arrays of different polymorphs or crystal forms using isothermic crystallization (FIG. 3A), temperature-mediated crystallization (FIG. 3B), and evaporative crystallization (FIG. 3C).

FIG. 4 relates to the Example and is a Raman intensity as a function of wave number for representative glycine crystals grown in under varying solvent and crystallization additive conditions as discussed in the Example: (A1) pure water, (B1) 4 v/o acetic acid, (C1) 6 v/o sulfuric acid, (D1) 0.1 wt % Triton X-100 and (F1) 0.1 wt % DL-serine.

### 6. DETAILED DESCRIPTION OF THE INVENTION

As an alternate approach to traditional methods for discovery of new solid-forms and discovery of conditions relating to formation, inhibition of formation, or dissolution of solid-forms, applicants have developed high-throughput methods to produce and screen hundreds, thousands, to hundreds of thousands of samples per day. The array technology described herein is a high-throughput approach that can be used to generate large numbers (greater than 10, more typically greater than 50 or 100, and more preferably 1000 or greater samples) of parallel small-scale solid-form experiments (e.g., crystallizations) for a given compound-of-interest, typically, less than about 1 g of the compound-of-interest, preferably, less than about 100 mg, more preferably, less than about 25 mg, even more preferably, less than about 1 mg, still more preferably less than about 100 micrograms, and optimally less than about 100 nanograms of the compound-of-interest. These methods are useful to optimize, select, and discover new, solid-forms having enhanced properties. The methods are also useful to discover compositions or conditions that promote formation of solid-forms with desirable properties. The methods are further useful to discover compositions or conditions that inhibit, prevent, or reverse formation of solid-forms.

In the preferred embodiment, the crystal forms are prepared in an array of sample sites, such as a 24, 48 or 96-well plate or more. Each sample in the array comprises a mixture of a compound-of-interest and at least one other component. The array is then subject to a set of processing parameters. Examples of processing parameters that can be varied to form different solid-forms include adjusting the temperature; adjusting the time; adjusting the pH; adjusting the amount or the concentration of the compound-of-interest; adjusting the amount or the concentration of a component; component identity (adding one or more additional components); adjusting the solvent removal rate; introducing of a nucleation event; introducing of a precipitation event; controlling evaporation of the solvent (e.g., adjusting a value of pressure or adjusting the evaporative surface area); and adjusting the solvent composition.

After processing, the contents of each sample in the processed array is typically analyzed initially for physical or structural properties, for example, the likelihood of crystal formation is assessed by turbidity, using a device such as a spectrophotometer. But a simple visual analysis can also be conducted including photographic analysis. Whether the detected solid is crystalline or amorphous can then be determined. More specific properties of the solid can then be measured, such as polymorphic form, crystal habit, particle size distribution, surface-to-volume ratio, and chemical and physical stability etc. Samples containing bioactive solids can be screened to analyze properties, such as altered bioavailability and pharmacokinetics. Bioactive solid-forms can be screened in vitro for their pharmacokinetics, such as absorption through the gut (for an oral preparation), skin (for transdermal application), or mucosa (for nasal, buccal, vaginal or rectal preparations), solubility, degradation or clearance by uptake into the reticuloendothelial system (“RES”) or excretion through the liver or kidneys following administration, then tested in vivo in animals. Testing can be done simultaneously or sequentially.

The methods and systems are widely applicable for different types of substances (compounds-of-interest), including pharmaceuticals, dietary supplements, alternative medicines, nutraceuticals, sensory compounds, agrochemicals, the active component of a consumer formulation, and the active component of an industrial formulation. Multiple solid-forms with desirable characteristics will typically be identified at each step of the testing, then subjected to additional testing.

6.1 System Design

The basic requirements for array and sample preparation and screening thereof are: (1) a distribution mechanism to add components and the compound-of-interest to separate sites, for example, on an array plate having sample wells or sample tubes. Preferably, the distribution mechanism is automated and controlled by computer software and can vary at least one addition variable, e.g., the identity of the component(s) and/or the component concentration, more preferably, two or more variables. Such material handling technologies and robotics are well known to those skilled in the art. Of course, if desired, individual components can be placed at the appropriate sample site manually. This pick and place technique is also known to those skilled in the art. And (2) a screening mechanism to test each sample to detect a change in physical state or for one or more properties. Preferably, the testing mechanism is automated and driven by a computer. Preferably, the system further comprises a processing mechanism to process the samples after component addition. Optionally, the system can have a processing station the process the samples after preparation.

A number of companies have developed array systems that can be adapted for use in the invention disclosed herein. Such systems may require modification, which is well within ordinary skill in the art. Examples of companies having array systems include Gene Logic of Gaithersburg, Md. (see U.S. Pat. No. 5,843,767 to Beattie), Luminex Corp., Austin, Tex., Beckman Instruments, Fullerton, Calif., MicroFab Technologies, Plano, Tex., Nanogen, San Diego, Calif., and Hyseq, Sunnyvale, Calif. These devices test samples based on a variety of different systems. All include thousands of microscopic channels that direct components into test wells, where reactions can occur. These systems are connected to computers for analysis of the data using appropriate software and data sets. The Beckman Instruments system can deliver nanoliter samples of 96 or 384-arrays, and is particularly well suited for hybridization analysis of nucleotide molecule sequences. The MicroFab Technologies system delivers sample using inkjet printers to aliquot discrete samples into wells. These and other systems can be adapted as required for use herein. For example, the combinations of the compound-of-interest and various components at various concentrations and combinations can be generated using standard formulating software (e.g., Matlab software, commercially available from Mathworks, Natick, Mass.). The combinations thus generated can be downloaded into a spread sheet, such as Microsoft EXCEL. From the spread sheet, a work list can be generated for instructing the automated distribution mechanism to prepare an array of samples according to the various combinations generated by the formulating software. The work list can be generated using standard programming methods according to the automated distribution mechanism that is being used. The use of so-called work lists simply allows a file to be used as the process command rather than discrete programmed steps. The work list combines the formulation output of the formulating program with the appropriate commands in a file format directly readable by the automatic distribution mechanism. The automated distribution mechanism delivers at least one compound-of-interest, such as a pharmaceutical, as well as various additional components, such as solvents and additives, to each sample well. Preferably, the automated distribution mechanism can deliver multiple amounts of each component. Automated liquid and solid distribution systems are well known and commercially available, such as the Tecan Genesis, from Tecan-US. RTP, North Carolina. The robotic arm can collect and dispense the solutions, solvents, additives, or compound-of-interest form the stock plate to a sample well or sample tube. The process is repeated until array is completed, for example, generating an array that moves from wells at left to right and from top to bottom in increasing polarity or non-polarity of solvent. The samples are then mixed. For example, the robotic arm moves up and down in each well plate for a set number of times to ensure proper mixing.

Liquid handling devices manufactured by vendors such as Tecan, Hamilton and Advanced Chemtech are all capable of being used in the invention. A prerequisite for all liquid handling devices is the ability to dispense to a sealed or sealable reaction vessel and have chemical compatibility for a wide range of solvent properties. The liquid handling device specifically manufactured for organic syntheses are the most desirable for application to crystallization due to the chemical compatibility issues. Robbins Scientific manufactures the Flexchem reaction block which consists of a Teflon reaction block with removable gasketed top and bottom plates. This reaction block is in the standard footprint of a 96-well microtiter plate and provides for individually sealed reaction chambers for each well. The gasketing material is typically Viton, neoprene/Viton; or Teflon coated Viton, and acts as a septum to seal each well. As a result, the pipetting tips of the liquid handling system need to have septum-piercing capability. The Flexchem reaction vessel is designed to be reusable in that the reaction block can be cleaned and reused with new gasket material.

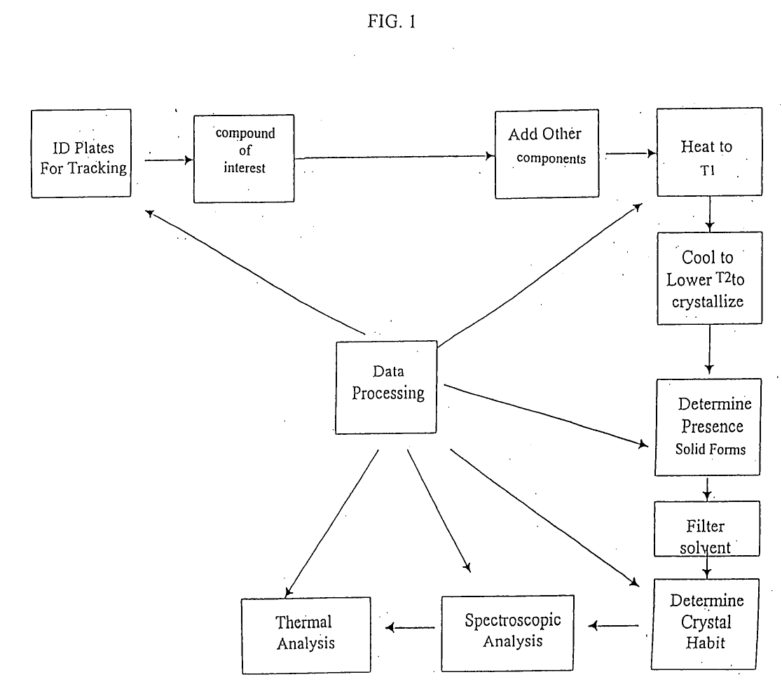

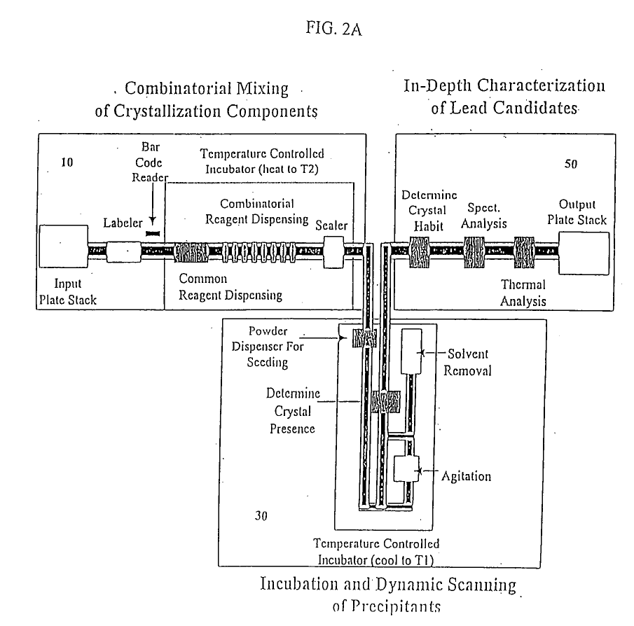

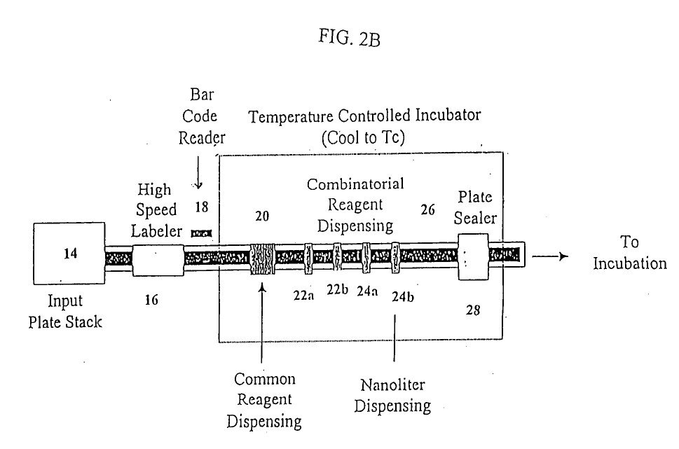

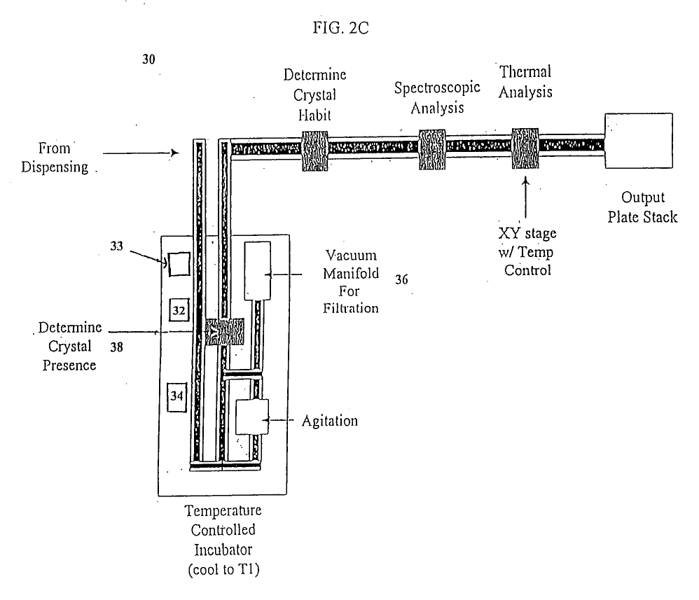

The schematic process for the preferred process is shown in FIGS. 1 and 2A-2C. The system consists of a series of integrated modules, or workstations. These modules can be connected directly, through an assembly-line approach, using conveyor belts, or can be indirectly connected by human intervention to move substances between modules.

One embodiment of the invention is depicted schematically in Scheme 1. As shown, plates are identified for tracking. Next, the compound-of-interest is added followed by various other components, such as solvents and additives. Preferably, the compound-of-interest and all components are added by an automated distribution mechanism. The array of samples is then heated to a temperature (T1), preferably to a temperature at which the active component is completely in solution. The samples are then cooled, to a lower temperature T2, usually for at least one hour. If desired, nucleation initiators such as seed crystals can be added to induce nucleation or an anti solvent can be added to induce precipitation. The presence of solid-forms is then determined, for example, by optical detection, and the solvent removed by filtration or evaporation. The crystal properties, such as polymorph or habit can then determined using techniques such as Raman, melting point, x-ray diffraction, etc., with the results of the analysis being analyzed using an appropriate data processing system.

6.2 Preparing Arrays

An array can be prepared, processed, and screened as follows. The first step comprises selecting the component sources, preferably, at one or more concentrations. Preferably, at least one component source can deliver a compound-of-interest and one can deliver a solvent. Next, adding the compound-of-interest and components to a plurality of sample sites, such as sample wells or sample tubes on a sample plate to give an array of unprocessed samples. The array can then be processed according to the purpose and objective of the experiment, and one of skill in the art will readily ascertain the appropriate processing conditions. Preferably, the automated distribution mechanism as described above is used to distribute or add components.

6.3 Processing Arrays

The array be processed according to the design and objective of the experiment. One of skill in the art will readily ascertain the appropriate processing conditions. Processing includes mixing; agitating; heating; cooling; adjusting the pressure; adding additional components, such as crystallization aids, nucleation promoters, nucleation inhibitors, acids, or bases, etc.; stirring; milling; filtering; centrifuging, emulsifying, subjecting one or more of the samples to mechanical stimulation; ultrasound; or laser energy; or subjection the samples to temperature gradient or simply allowing the samples to stand for a period of time at a specified temperature. A few of the more important processing parameters are elaborated below.

6.3.1 Temperature

In some array experiments, processing will comprise dissolving either the compound-of-interest or one or more components. Solubility is commonly controlled by the composition (identity of components and/or the compound-of-interest) or by the temperature. The latter is most common in industrial crystallizers where a solution of a substance is cooled from a state in which it is freely soluble to one where the solubility is exceeded. For example, the array can be processed by heating to a temperature (T1), preferably to a temperature at which the all the solids are completely in solution. The samples are then cooled, to a lower temperature (T2). The presence of solids can then determined. Implementation of this approach in arrays can be done on an individual sample site basis or for the entire array (i.e., all the samples in parallel). For example, each sample site could be warmed by local heating to a point at which the components and the compound-of-interest are dissolved. This step is followed by cooling through local thermal conduction or convection. A temperature sensor in each sample site can be used to record the temperature when the first crystal or precipitate is detected. In one embodiment, all the sample sites are processed individually with respect to temperature and small heaters, cooling coils, and temperature sensors for each sample site are provided and controlled. This approach is useful if each sample site has the same composition and the experiment is designed to sample a large number of temperature profiles to find those profiles that produce desired solid-forms. In another embodiment, the composition of each sample site is controlled and the entire array is heated and cooled as a unit. The advantage of the latter approach is that much simpler heating, cooling, and controlling systems can be utilized. Alternatively, thermal profiles are investigated by simultaneous experiments on identical array stages. Thus, a high-throughput matrix of experiments in both composition and thermal profiles can be obtained by parallel operation.

Typically, several distinct temperatures are tested during crystal nucleation and growth phases. Temperature can be controlled in either a static or dynamic manner. Static temperature means that a set incubation temperature is used throughout the experiment. Alternatively, a temperature gradient can be used. For example, the temperature can be lowered at a certain rate throughout the experiment. Furthermore, temperature can be controlled in a way as to have both static and dynamic components. For example, a constant temperature (e.g., 60° C.) is maintained during the mixing of crystallization reagents. After mixing of reagents is complete, controlled temperature decline is initiated (e.g. 60° C. to about 25° C. over 35 minutes).

Stand-alone devices employing Peltier-effect cooling and joule-heating are commercially available for use with microtiter plate footprints. A standard thermocycler used for PCR, such as those manufactured by MJ Research or PE Biosystems, can also be used to accomplish the temperature control. The use of these devices, however, necessitates the use of conical vials of conical bottom micro-well plates. If greater throughput or increased user autonomy is required, then full-scale systems such as the advanced Chemtech Benchmark Omega 96™ or Venture 596 ™ would be the platforms of choice. Both of these platforms utilize 96-well reaction blocks made from Teflon™. These reaction blocks can be rapidly and precisely controlled from −70 to 150° C. with complete isolation between individual wells. Also, both systems operate under inert atmospheres of nitrogen or argon and utilize all chemically inert liquid handling elements. The Omega 496 system has simultaneous independent dual coaxial probes for liquid handling, while the Venture 596 system has 2 independent 8-channel probe heads with independent z-control. Moreover, the Venture 596 system can process up to 10,000 reactions simultaneously. Both systems offer complete autonomy of operation.

6.3.2 Time

Array samples can be incubated for various lengths of time (e.g., 5 minutes, 60 minutes, 48 hours, etc.). Since phase changes can be time dependent, it can be advantageous to monitors arrays experiments as a function of time. In many cases, time control is very important, for example, the first solid-form to crystallize may not be the most stable, but rather a metastable form which can then convert to a form stable over a period of time. This process is called “ageing”. Ageing also can be associated with changes in crystal size and/or habit. This type of ageing phenomena is called Ostwald ripening.

6.3.3 pH

The pH of the sample medium can determine the physical state and properties of the solid phase that is generated. The pH can be controlled by the addition of inorganic and organic acids and bases. The pH of samples can be monitored with standard pH meters modified according to the volume of the sample.

6.3.4 Concentration

Supersaturation is the thermodynamic driving force for both crystal nucleation and growth and thus is a key variable in processing arrays. Supersaturation is defined as the deviation from thermodynamic solubility equilibrium. Thus the degree of saturation can be controlled by temperature and the amounts or concentrations of the compound-of-interest and other components. In general, the degree of saturation can be controlled in the metastable region, and when the metastable limit has been exceeded, nucleation will be induced.

The amount or concentration of the compound-of-interest and components can greatly effect physical state and properties of the resulting solid-form. Thus, for a given temperature, nucleation and growth will occur at varying amounts of supersaturation depending on the composition of the starting solution. Nucleation and growth rate increases with increasing saturation, which can affect crystal habit. For example, rapid growth must accommodate the release of the heat of crystallization. This heat effect is responsible for the formation of dendrites during crystallization. The macroscopic shape of the crystal is profoundly affected by the presence of dendrites and even secondary dendrites. The second effect that the relative amounts compound-of-interest and solvent has is the chemical composition of the resulting solid-form. For example, the first crystal to be formed from a concentrated solution is formed at a higher temperature than that formed from a dilute solution. Thus, the equilibrium solid phase is that from a higher temperature in the phase diagram. Thus, a concentrated solution may first form crystals of the hemihydrate when precipitated from aqueous solution at high temperature. The dihydrate may, however, be the first to form when starting with a dilute solution. In this case, the compound-of-interest/solvent phase diagram is one in which the dihydrate decomposes to the hemihydrate at a high temperature. This is normally the case and holds for commonly observed solvates.

6.3.5 Identity of the Components

The identity of the components in the sample medium has a profound effect on almost all aspects of solid formation. Component identity will affect (promote or inhibit) crystal nucleation and growth as well as the physical state and properties of the resulting solid-forms. Thus, a component can be a substance whose intended effect in an array sample is to induce, inhibit, prevent, or reverse formation of solid-forms of the compound-of-interest. A component can direct formation of crystals, amorphous-solids, hydrates, solvates, or salt forms of the compound-of-interest. Components also can affect the internal and external structure of the crystals formed, such as the polymorphic form and the crystal habit. Examples of components include, but are not limited to, excipients; solvents; salts; acids; bases; gases; small molecules, such as hormones, steroids, nucleotides, nucleosides, and aminoacids; large molecules, such as oligonucleotides, polynucleotides, oligonucleotide and polynucleotide conjugates, proteins, peptides, peptidomimetics, and polysaccharides; pharmaceuticals; dietary supplements; alternative medicines; nutraceuticals; sensory compounds; agrochemicals; the active component of a consumer formulation; and the active component of an industrial formulation; crystallization additives, such as additives that promote and/or control nucleation, additives that affect crystal habit, and additives that affect polymorphic form; additives that affect particle or crystal size; additives that structurally stabilize crystalline or amorphous solid-forms; additives that dissolve solid-forms; additives that inhibit crystallization or solid formation; optically-active solvents; optically-active reagents; and optically-active catalysts.

6.3.6 Control of Solvent-Removal Rate

Control of solvent removal is intertwined with control of saturation. As the solvent is removed, the concentration of the compound-of-interest and less-volatile components becomes higher. And depending on the remaining composition, the degree of saturation will change depending on factors, such as the polarity and viscosity of the remaining composition. For example, as a solvent it removed, the concentration of the component-of-interest can rise until the metastable limit is reached and nucleation and crystal growth occur. The rate of solvent removal can be controlled by temperature and pressure and the surface area under which evaporation can occur. For example, solvent can be removed by distillation at a predefined temperature and pressure, or the solvent can be removed simply by allowing the solvent to evaporate at room temperature.

6.3.7 Inducing Solid-Formation by Introducing a Nucleation or Precipitation Event

Once an array is prepared, solid formation can be induced by introducing a nucleation or precipitation event. In general, this involves subjecting a supersaturated solution to some form of energy, such as ultrasound or mechanical stimulation or by inducing supersaturation by adding additional components.

6.3.7.1 Introducing a Nucleation Event

Crystal nucleation is the formation of a crystal solid phase from a liquid, an amorphous phase, a gas, or from a different crystal solid phase. Nucleation sets the character of the crystallization process and is therefore one of the most critical components in designing commercial crystallization processes and the crystallizer's design and operation, (1993) The Encyclopedia of Chemical Technology, 7 Kirk-Othomer (4^{th} ed. at 692), incorporated herein by reference. So called primary nucleation can occur by heterogenous or homogeneous mechanisms, both of which involve crystal formation by sequential combining of crystal constituents. Primary nucleation does not involve existing crystals of the compound-of-interest, but results from spontaneous formation of crystals. Primary nucleation can be induced by increasing the saturation over the metastable limit or, when the degree of saturation is below the metastable limit, by nucleation. Nucleation events include mechanical stimulation, such as contact of the crystallization medium with the stirring rotor of a crystallizer and exposure to sources of energy, such as acoustic (ultrasound), electrical, or laser energy (e.g., see Garetz et al., 1996 Physical review Letters 77:3475. Primary nucleation can also be induced by adding primary nucleation promoters, that is substances other than a solid-form of the compound-of-interest. Additives that decrease the surface energy of the compound to be crystallized can induce nucleation. A decrease in surface energy favors nucleation, since the barrier to nucleation is caused by the energy increase upon formation of a solid-liquid surface. Thus, in the current invention, nucleation can be controlled by adjusting the interfacial tension of the crystallizing medium by introducing surfactant-like molecules either by pre-treating the sample tubes or sample wells or by direct addition. The nucleation effect of surfactant molecules is dependent on their concentration and thus this parameter should be carefully controlled. Such tension adjusting additives are not limited to surfactants. Many compounds that are structurally related to the compound-of-interest can have significant surface activity. Other heterogeneous nucleation inducing additives include solid particles of various substances, such as solid-phase excipients or even impurities left behind during synthesis or processing of the compound-of-interest.

Similarly, inorganic crystals on specifically functionalized self-assembled monolayers (SAMs) have also been demonstrated to induce nucleation by Wurm, et al.,1996, J. Mat. Sci. Lett. 15:1285 (1996). Nucleation of organic crystals such as 4-hydroxybenzoic acid monohydrate on a 4-(octyldecyloxy)benzoic acid monolayer at the air-water interface has been demonstrated by Weissbuch, et al. 1993 J. Phys. Chem. 97:12848 and Weissbuch, et al., 1995 J. Phys. Chem. 99:6036. Nucleation of ordered two dimensional arrays of proteins on lipid monolayers has been demonstrated by Ellis et al., 1997, J. Struct. Biol. 118:178.

Secondary nucleation involves treating the crystallizing medium with a secondary nucleation promoter, that is a solid-form, preferably a crystalline form of the compound-of-interest. Direct seeding of samples with a plurality of nucleation seeds of a compound-of-interest in various physical states provides a means to induce formation of different solid-forms. In one embodiment, particles are added to the samples. In another, nanometer-sized crystals (nanoparticles) of the compound-of-interest are added to the samples.

6.3.7.2 Introducing a Precipitation Event

The term precipitation is usually reserved to describe the formation of an amorphous solid or semi-solid from a solution phase. Precipitation can be induced in much the same way as discussed above for nucleation the difference being that an amorphous rather than a crystalline solid is formed. Addition of a nonsolvent to a solution of a compound-of-interest can be used to precipitate a compound. The nonsolvent rapidly decreases the solubility of the compound in solution and provides the driving force to induce solid precipitate. This method generally produces smaller particles (higher surface area) than by changing the solubility in other ways, such as by lowering the temperature of a solution. The invention provides means to identify the optimal solvents and solvent concentrations for providing an optimal solid-form or for preventing formation or inducing salvation of a solid-form. The invention can be used to greatly speed the process of identifying useful precipitation solvents.

Precipitation can also be induced by changing the composition of the compound-of-interest such that it is no longer as soluble or is insoluble. For example, by addition of acidic components or basic components that react to form a salt with the compound-of-interest, the salt being less soluble than the original compound or insoluble. Compounds-of-interest that are basic in nature are capable of forming a wide variety of salts with various inorganic and organic acids. When the compound-of-interest is a pharmaceutical, preferably, the acids used are those that form salts comprising pharmacologically acceptable anions including, but not limited to, acetate, benzenesulfonate, benzoate, bicarbonate, bitartrate, bromide, calcium edetate, camsylate, carbonate, chloride, bromide, iodide, citrate, dihydrochloride, edetate, edisylate, estolate, esylate, fumarate, gluceptate, gluconate, glutamate, glycollylarsanilate, hexylresorcinate, hydrabamine, hydroxynaphthoate, isethionate, lactate, lactobionate, malate, maleate, mandelate, mesylate, methylsulfate, muscate, napsylate, nitrate, panthothenate, phosphate/diphosphate, polygalacturonate, salicylate, stearate, succinate, sulfate, tannate, tartrate, teoclate, triethiodide, and pamoate (i.e., 1,1′-methylene-bis-(2-hydroxy-3-naphthoate)). Compounds-of-interest that include an amino moiety also can form pharmaceutically-acceptable salts with various amino acids, in addition to the acids mentioned above. Compounds-of-interest that are acidic in nature are capable of forming base salts with various cations. Examples of such salts include alkali metal or alkaline earth metal salts and, particularly, calcium, magnesium, sodium, lithium, zinc, potassium, and iron salts, as well as salts of basic organic compounds, such as amines, for example N-methylglucamine and TRIS (tris-hydroxymethyl aminomethane).

6.3.8 Solvent Composition

The use different solvents or mixtures of solvents will influence the solid-forms that are generated. Solvents may influence and direct the formation of the solid phase through polarity, viscosity, boiling point, volatility, charge distribution, and molecular shape. In a preferred embodiment, solvents that are generally accepted within the pharmaceutical industry for use in manufacture of pharmaceuticals are used in the arrays. Various mixtures of those solvents can also be used. The solubilities of the compound-of-interest is high in some solvents and low in others. Solutions can be mixed in which the high-solubility solvent is mixed with the low-solubility solvent until solid formation is induced. Hundreds of solvents or solvent mixtures can be screened to find solvents or solvent mixtures that induce or inhibit solid-form formation. Solvents include, but are not limited to, aqueous based solvents such as water or aqueous acids, bases, salts, buffers or mixtures thereof and organic solvents, such as protic, aprotic, polar or non-polar organic solvents.

6.4 Screening Arrays for the Presence or Absence of Solid-Forms and Further Analysis of Detected Solid-Forms

In certain embodiments, after processing, samples can be analyzed to detect the presence or absence of solid-forms, and any solid-forms detected can be further analyzed, e.g., to characterize the properties and physical state.

Advantageously, samples in commercially available microtiter plates, can be screened for the presence or absence of solids (e.g., precipitates or crystals) using automated plate readers. Automated plate readers can measure the extent of transmitted light across the sample. Diffusion (reflection) of transmitted light indicates the presence of a solid-form. Visual or spectral examination of these plates can also be used to detect the presence of solids. In yet another method to detect solids, the plates can be scanned by measuring turbidity.

If desired, samples containing solids can be filtered to separate the solids from the medium, resulting in an array of filtrates and an array of solids. For example, the filter plate comprising the suspension is placed on top of a receiver plate containing the same number of sample wells, each of which corresponds to a sample site on the filter plate. By applying either centrifugal or vacuum force to the filter plate over receiver plate combination, the liquid phase of the filter plate is forced through the filter on the bottom of each sample well, into the corresponding sample well of the receiver plate. A suitable centrifuge is available commercially, for example, from DuPont, Wilmington, Del. The receiver plate is designed for analysis of the individual filtrate samples.

After a solid is detected it can be further analyzed to define its physical state and properties. In one embodiment, on-line machine vision technology is used to determine both the absence/presence of crystals as well as detailed spatial and morphological information. Crystallinity can be assessed and distinguished from amorphous solids automatically using plate readers with polarized filter apparatus to measure the total light to determine crystal birefringence; crystals turn polarized light, while amorphous materials absorb the light. Plate readers are commercially available. It is also possible to monitor turbidity or birefringence dynamically throughout the crystal forming process. True polymorphs, solvates, and hydrates, can be tested by x-ray Powder Diffraction (XRPD) (angles of diffracted laser light can be used to determine whether true polymorph(s) have been formed). Different crystalline forms can be determined by differential scanning calorimetry (DSC) and Thermoraphic Gravimetric (TG) analysis.

6.4.1 Raman and Infrared Spectroscopy of Solids

Raman and Infrared spectroscopy are useful methods for analysis of solids, one advantage being that it can be performed without sample dissolution. The infrared and near infrared spectrum are extremely sensitive to structure and conformation. The method involves grinding the sample and suspending it in Nujor or grinding the sample with KBr and pressing this mixture into a disc. This preparation is then placed in the near infrared or infrared sample beam and the spectrum is recorded.

Raman and Infrared spectroscopy are also useful in the investigation of polymorphs in the solid state. For example, polymorphs A and B of tolbutamide give different infrared spectra (Simmons et al., 1972). It is clear that there are significant differences between the spectra of the polymorphs.

6.4.2 Second Harmonic Generation (SGH)

Symmetry lowering in host-additive systems (crystals incorporating guest molecules, e.g., solvates), such as a loss of inversion, glide, or twofold screw symmetry, which would introduce polarity into the crystal, can be probed by non-linear optical effects, such as second harmonic generation, which is active in a noncentrosymmetric crystalline forms. For a comprehensive review on second harmonic generation see Corn et al., 1994 Chem. Rev. 94:107-125.

6.4.3 X-Ray Diffraction

The x-ray crystallography technique, whether performed using single crystals or powdered solids, concerns structural analysis and is well suited for the characterization of polymorphs and solvates as well as distinguishing amorphous from crystalline solids. In the most favorable cases, it can lead to a complete determination of the structure of the solid and the determination of the packing relationship between individual molecules in the solid.

Single crystal x-ray diffraction is the preferred analytical technique to characterize crystals generated according to the arrays and methods of the invention. The determination of the crystal structure requires the determination of the unit-cell dimensions and the intensities of a large fraction of the diffracted beams from the crystal.

The first step is selection of a suitable crystal. Crystals should be examined under a microscope and separated into groups according to external morphology or crystal habit. For a complete study, each crystal of a completely different external morphology should be examined.

Once the crystals have been separated according to shape, the best crystal of the first group should be mounted on a goniometer head with an adhesive such as glue.

The unit cell dimension are then determined by photographing the mounted crystal on a precession camera. The unit cell parameters are determined from the precession photograph by measuring the distance between the rows and columns of spots and the angle between a given row and column. This is done for three different orientations of the crystal, thus allowing determination of the unit cell dimensions.

The intensities of the diffracted radiation are most conveniently measured using an automated diffractometer that is a computer-controlled device that automatically records the intensities and background intensities of the diffracted beams on a magnetic tape. In this device, the diffracted beam is intercepted by a detector, and the intensity is recorded electronically.

The diffraction data are then converted to electron density maps using standard techniques, for example, the DENZP program package (Otwinowski, et al., Methods in Enzymology 276 (1996)). Software packages, such as XPLOR (A. Brunger, X-PLOR Manual, Yale University), are available for interpretation of the data. For more details, see Glusker, J. P. and Trueblood, K. N. Crystal Structure Analysis”, Oxford University Press, 1972.

X-ray Powder Diffraction can also be used. The method that is usually used is called the Debye-Scherrer method (Shoemaker and Garland, 1962). The specimen is mounted on a fiber and placed in the Debye-Scherrer powder camera. This camera consists of an incident-beam collimator, a beam stop, and a circular plate against which the film is placed. During the recording of the photograph, the specimen is rotated in the beam. Because the crystallites are randomly oriented, at any given Bragg angle, a few particles are in diffracting position and will produce a powder line whose intensity is related to the electron density in that set of planes.

This method, along with precession photography, can be used to determine whether crystals formed under different conditions are polymorphs or merely differ in crystal habit. To measure a powder pattern of a crystal or crystals on a Debye-Scherrer camera, one grinds the sample to a uniform size (200-300 mesh). The sample is then placed in a 0.1- to 0.5-mm-diameter glass capillary tube made of lead-free glass. Commercially made capillary tubes with flared ends are available for this purpose. The capillary tube is placed on a brass pin and inserted into the pin-holder in a cylindrical Debye-Scherrer powder camera. The capillary tube is aligned so that the powdered sample remains in the x-ray beam for a 360° rotation. Film is then placed in the camera, and the sample is exposed to CuK_{α} x-rays. The film is then developed and the pattern is compared to the pattern from other crystals of the same substance. If the patterns are identical the crystals have the same internal structure. If the patterns are different, then the crystals have a different internal structure and are polymorphs.

6.4.4 Image-Analysis Techniques

Image-analysis techniques are powerful techniques that allow surface characterization of various types of materials. The images obtained using these various techniques allow one to obtain information about a sample that would not otherwise be available using conventional techniques. When one of these techniques is used in conjunction with the others, one could obtain complementary images or data that would aid in elucidating the structure, property, or behavior of a material, for example, crystal habit. Depending on the type of sample to be characterized, one may incorporate modifications into a typical setup or adjust the various experimental parameters to allow optimal characterization of the sample. These various techniques are discussed in more details below.

6.4.4.1 Microscopy and Photomicrography

This method of optical-image analysis involves the observation of the behavior of a crystal on a microscope (Kuhuert-Brandstatter, 1971). Crystals are usually placed on a microscope slide and covered with a cover slip. However, sometimes a steel ring with input and output tubes is used to control the atmosphere.

The microscope slide is often placed on a “hot stage,” a commercially available device for heating crystals while allowing observation with a microscope. The heating rate of crystals on a hot stage is usually constant and controlled with the help of a temperature programmer.

Crystals are often photographed during heating. Photography is helpful because for slid-state reactions taking weeks to complete it is sometimes difficult to remember the appearance of a crystal during the entire reaction. Obviously, photography permanently preserves the details of the reaction.

The following types of behavior are of particular interest to the solid-state chemist:
1. The loss of solvent of crystallization.
2. Sublimation of the crystal—the crystal slowly disappears and condenses on the cover slip.
3. Melting and resolidification, indicating a phase change (polymorphic transformation) or solid-state reaction.
4. Chemical reaction characterized by a visible change in the appearance of the crystal.

The detection of loss of solvent of crystallization and phase or polymorphic transformations is important to the solid-state chemist, since crystals exhibiting this behavior can have different reactivity and different bioavailability. Sublimation, while not a solid-state reaction, can cause confusion if one is unaware that it can occur.

6.4.4.2 Electron Microscopy

Electron microscopy, which can be used as an optical-imaging technique, is a powerful tool for studying the surface properties of crystals. High-resolution election microscopy can be used to visualize lattice fringes in inorganic compounds, but its usefulness for visualization of lattice fringes in organic compounds is so far unproven. Nevertheless, electron micrographs of organic crystals allow the examination of the crystal surface during reaction. Electron microscopy is particularly useful for studying the affects of structural imperfections and dislocations on solid-state organic reactions. For example, the surface photooxidation of anthracene is obvious from electron micrographs taken at a magnification of 10,000 (Thomas, 1974). Even more interesting is the use of electron microscopy, sometimes in conjunction with optical microscopy, to study the effects of dislocations and various kinds of defects on the nucleation of product phase during a solid state reaction.

Electron microscopy is also quite useful for the studies of the effect of crystal size on desolvation reactions. Electron micrographs have significantly more depth of field than optical micrographs, so that the average crystal size can be more easily determined using them.

Scanning electron microscopy (SEM) is well suited for examining topography such as fracture surfaces. It allows convenient preparation of specimen to be imaged for analyzing the microstructure of materials. Using the backscattered electron mode of SEM, one can obtain both topographic, crystallographic, and composition information. P. E. J. Flewitt & R. K. Wilk, Physical Methods for Materials Characterization, Institute of Physics Publishing, London (1994). Combination with computer automation has facilitate instrument control and image processing.

Transmission electron microscope (TEM) is one of the most powerful instruments for microstructure analysis of materials. In TEM, the two modes of viewing images are bright field and dark field images. These two modes yields essential microstructural information from a specimen. For example, in the bright field mode, one can observe dislocations in various types of materials because these dislocations produce crystal lattice displacements that produce images. When the first high resolution images were obtained using TEM, atom positions in two dimensional lattices were determined from the observed intensity peaks. Also, under carefully controlled conditions, TEM provides crystallographic information such as the spacing of crystal lattice planes in a specimen. Id.

Other microscopy techniques that may be used in conjunction with the above techniques to characterize crystals are optical microscopy methods such as near-field scanning optical microscopy (NSOM or SNOM) and far-field scanning optical microscopy. These techniques, which are discussed below, allow one to characterize materials by scanning the sample to obtain a sample's topographic image. With AFM, one can obtain a three-dimensional image of a surface with atomic resolution. Micro-Thermal Analysis, which provides a thermal conductivity image of a sample, provides additional information about a sample such as phase transitions.

6.4.4.3 Near Field Scanning Optical Microscopy

Near field scanning optical microscopy (NSOM or SNOM), an image analysis technique, is a scanning probe microscopy that permits optical imaging with spatial resolution beyond the diffraction limit. Using NSOM, it has been possible to achieve a resolution as high as about 50 nm, the highest optical resolution attained with visible light. NSOM has been used to characterize the optical and topographical features of materials such as polymer blends, composites, biological materials (using wet-cell NSOM) such as proteins, monolayers, and single crystals. See D. W. Pohl, “Scanning rear-field optical microscopy,” Advances in Optical and Electron Microscopy, 12, C. J. R. Sheppard and T. Mulvey, Eds. (Adademic Press, London, 1990); E. Betzig and J. K. Trautman, Science, 257, 189 (1992); McDaniel et al., “Local Characterization of a two-dimensional photonic crystal,” Phys. Rev. B, 55, 10878 (1998)

NSOM is a very useful technique in that it can be combined with conventional spectroscopic and imaging techniques (e.g., fluorescence, absorption, or polarization spectroscopy) to produce images having extremely high resolution. It offers the potential of resolving spectroscopic components of heterogeneous materials on a submicron length scale. This allows elucidation of the relationship between spectroscopic (optical) properties and microscopic structure (topography). The high resolution is achieved by avoiding diffraction effects through the use of sub-wavelength light source maintained in the near-field of the sample surface. Typically, the fiber tip is held tens of nanometers above the sample surface. Thus, the light is forced to interact with the sample before the light undergoes diffraction, and sub-diffraction optical (“super”) resolution is obtained. The topographic image obtained is similar to that obtained using a conventional contact atomic force microscope.

In a typical NSOM set-up, a single mode fiber is heated with a laser such as a CO, laser to the working point and drawn to a fine point (using a micropipette puller) measuring about 50-100 nm in diameter. The tip is then evaporatively coated with aluminum to form a subwavelength aperture at the apex of the fiber tip. The aluminum coating is used to prevent light from leaking out of the sides of the tip taper. Using the NSOM tip, one can illuminate a subwavelength sized region (transmission mode) or to collect radiation emitted from a submicron sized area (collection mode) of a sample. The spatial extent of the illuminated region can be substantially smaller than that which can be achieved with conventional lenses.

NSOM has been used to obtain images of optical transmission, fluorescence emission, and birefringence from thin transparent samples. In one particular method of characterizing a sample, laser light leaves the NSOM tip and irradiates the sample thereby causing the sample molecules to jump to an excited state. The fluorescence subsequently emitted by the sample is collected by a high numerical aperture objective. The sample preferably must be thin enough so that sufficient amount of light can be detected. This is so because the molecules on or near the surface affect the intensity of the detected light more significantly than the molecules buried deeper from the surface. Ideally, the samples are prepared to produce thin films on glass microscope cover slips or their equivalent. The sample surface area should be about 1-1.5 cm in diameter.

6.4.4.4 Far Field Scanning Optical Microscopy

As opposed to NSOM, far field microscopy, which can also be used as an image analysis technique, is limited by the diffraction of light. In far field microscopy, the distance between the observer and the light source is more than a the wavelength of the emitted light while the reverse is true in near-field microscopy. Also, in conventional far field microscopy such as a conventional microscope, one obtains the entire image at once. Thus, an image obtained using it has a resolution which is limited by the wavelength of light. But a method has been developed that allows one to obtain three-dimensional structural information on a length scale well below the Rayleigh length using conventional far-field optics. By spectrally selecting a single molecule with high-resolution laser spectroscopy and using a CCD camer to register the spatial distribution of the emitted photons in thee dimensions, one can resolve details in the specimen with sub-diffraction limited resolution in three dimensions. This technique has been proven to work with organic compounds such as pentacene in p-terphenyl at cryogenic temperatures. Van Oijeu, “Far-Field fluorescence microscopy beyond the diffraction limit,” J. Opt. Soc. Am, A, 16, 909 (1999).

6.4.4.5 Atomic-Force Microscopy

AFM is used in the characterization, an image analysis technique, of thick and thin films comprising materials ranging from organic materials, ceramics, composites, glasses, synthetic and biological membranes, metals, polymer, and semiconductors. AFM allows one to obtain a surface image with atomic resolution. It also allows measurement of the force in nano-Newton scale. AFM differs from conventional optical microscopy in that it allows one to obtain a three-dimensional image of the topography of a sample surface. See Atomic Force Microscopy/Scanning Tunneling Microscopy, Vol. 3, S. H. Cohen and M. L. Lightbody (eds.) Kluwer Academic/Plenum Publishers, New York (1998); Binnig et al., “Atomic Force Microscope,” Phys. Rev. Lett. 56, 930 (1986).

In a typical AFM, a sharp tip is scanned over a surface with feedback mechanisms that allow the piezoelectric scanners to maintain the tip at a constant force (to yield height information), or constant height (to yield force information) above the sample surface. The AFM head uses an optical detection system in which the tip is attached to the end of a cantilever. The tip-cantilever assembly is typically made of Si or Si₃N₄. In a typical AFM setup, a diode laser is focused unto the back of a reflective cantilever. As the tip scans the sample surface, bobbing up and down with the contours of the surface, the laser beam is deflected off the attached cantilever into a dual-element photodiode. The photodetector measures the difference in light intensities between the upper and lower photodetectors converts the difference to voltage. Feedback from the photodiode difference signal, using software control from the computer, allows the tip to maintain either a constant force or constant height above the sample.

There are different types of detection systems used. Interferometry is the most sensitive among the optical detection methods, but it is relatively more complicated than the now widely-used beam-bounce method. In the beam-bounce technique, the optical beam is reflected from the mirrored surface on the back side of the cantilever onto a position-sensitive photodetector. Another optical detection method makes use of the cantilever as one of the mirrors in the cavity of a diode laser. The movement of the cantilever affects the laser output, and this forms a basis for a motion detector.

Depending on the AFM design, scanners are used to translate either the sample beneath the cantilever or the cantilever over the sample. Either way, the local height of the sample can be measured. Three-dimensional topographical maps of the surface can be constructed by plotting the local sample height versus the horizontal probe tip position. AFM normally makes use of vibrational isolation to obtain a good scan.

6.4.5 Micro-Thermal Analysis

The operational principles of Micro-Thermal Analysis (Micro-TA) is based on atomic force microscopy (AFM). As mentioned above, AFM uses a tip/cantilever/laser/photodetector assembly to obtain a three dimensional map of the sample surface. One difference between the regular AFM and Micro-TA is that the latter uses as a probe that has a resistive heater at the tip. The most-widely used probe is made of Wollaston wire. When an electrical current flows through the probe, the tip heats up. The electrical resistance of the probe allows measurement of the tip temperature.

The simplest mode of operation is one where the probe's temperature is held constant and the electric power required to maintain the temperature is measured. The probe is then used to scan the sample surface in a contact AFM mode of operation. When the probe encounters a sample area that has a high thermal conductivity, more heat is lost from the tip to the sample than when a particular sample area being scanned has a low thermal conductivity. Thus, more electrical power is required to keep the temperature constant the higher the thermal conductivity of a sample area. One thus obtains a thermal conductivity map of a sample showing areas of high and low thermal conductivities. In a multi-component sample such as a given drug formulation, the thermal conductivity map allows one to visualize the various phases or phase transitions of the multi-component system based on their thermal or topographic properties. A melting process determined from the thermal map would aid in the identification of a compound or mixture such as a drug. This makes Micro-TA a highly useful tool for characterizing organic compounds including polymers. See Reading et al., “Thermal Analysis for the 21^{st} Century, American Laboratory, 30, 13 (1998); Price et al., “Micro-Thermal Analysis: A New Form of Analytical Microscopy,” Microscopy and Analysis, 65, 17 (1998).

6.4.6 Differential Thermal Analysis

Differential Thermal Analysis (DTA) is a method in which the temperature of the sample (Tₛ) is compared to the temperature of a reference compound (Tᵥ) as a function of increasing temperature. Thus, a DTA thermogram is a plot of ΔT=Tₛ−Tᵥ (temperature difference) versus T. The endotherms represent processes in which heat is absorbed, such as phase transitions and melting. The exotherms represent processes such as chemical reactions where heat is evolved. In addition, the area under a peak is proportional to the heat change involved. Thus, this method with proper calibration can be used to determine the heats (ΔH) of the various processes, the temperatures of processes such as melting, Tₘ, can be used as an accurate measure of the melting point.

There are a number of factors that can affect the DTA curve, including heating rate, atmosphere, the sample holder and thermocouple location, and the crystal size and sample packing. In general, the greater the heating rate the greater the transition temperature (i.e., Tₘ). An increased heating rate also usually causes the endotherms and exotherms to become sharper. The atmosphere of the sample affects the DTA curve. If the atmosphere is one of the reaction products, then increases in its partial pressure would slow down the reaction. The shape of the sample holder and the thermocouple locations can also affect the DTA trace. Thus, it is a good idea to only compare data measured under nearly identical conditions. The crystal size and packing of the sample has an important influence on all reactions of the type solid—solid+gas. In such reactions, increased crystal size (thus decreased surface area) usually decreases the rate of the reaction and increases the transition temperature.

An important type of differential thermal analysis is differential scanning calorimetry (DSC). Differential Scanning Calorimetry refers to a method very similar to DTA in which the ΔH of the reactions and phase transformations can be accurately measured. A DSC trace looks very similar to a DTA trace, and in a DSC trace the area under the curve is directly proportional to the enthalpy change. Thus, this method can be used to determine the enthalpies of various processes (Curtin et al., 1969).

6.4.7 Analytical Methods Requiring Dissolution of the Sample

While in some cases it is necessary to analyze the products of a solid-state reaction in the solid without dissolution, many of the most popular analytical methods of analysis require dissolution of the sample. These methods are useful for solid-state reactions if the reactants and products are stable in solution. For example, for solid-state reactions induced by heat or light, it is convenient to remove the heat or light, dissolve the sample, and analyze the products. In this section several important methods are reviewed and examples of their use in solid-state chemistry is discussed.

6.4.7.1 Ultraviolet Spectroscopy

Ultraviolet spectroscopy is very useful for studying the rates of solid-state reactions. Such studies require that the amount of reactant or product be measured quantitatively. Pendergrass et al. (1974) developed an ultraviolet method for the analysis of the solid-state thermal reaction of azotribenzoylmethane. In this reaction, the yellow (H1) thermally rearranges to the red (H2) and white (H3) forms in the solid state. All three compounds (H1, H2, and H3) have different chromophores, so that this reaction is amenable to analysis by ultraviolet spectroscopy. Pendergrass developed a matrix-algebra method for analyzing multi component mixtures by ultraviolet spectroscopy and used it to analyze the rate of the solid-state reaction under various conditions.

6.4.7.2 Nuclear Magnetic Resonance (NMR) Spectroscopy

The observation of NMR spectra requires that the sample be placed in a magnetic field where the normally degenerate nuclear energy levels are split. The energy of transition between these levels is then measured. In general, the proton magnetic resonance spectra are measured for quantitative analysis, although the spectra of other nuclei are also sometimes measured.

There are three important quantities measured in NMR spectroscopy: the chemical shift; the spin-spin coupling constant, and the area of the peak. The chemical shift is related to the energy of the transition between nuclei, the spin-spin coupling constant is related to the magnetic interaction between nuclei, and the area of the peak is related to the number of nuclei responsible for the peak. It is the area of the peak that is of interest in quantitative NMR analysis.

The ratio of the areas of the various peaks in proton NMR spectroscopy is equal to the ratio of protons responsible for these peaks. For multi component mixtures, the ratios of areas of peaks from each component are proportional both to the number of protons responsible for the peak and to the amount of the component. Thus, the addition of a known concentration of an internal standard allows the determination of the concentrations of the species present. Unfortunately, area measurement is subject to several errors and the accuracy of this method is seldom better than 1 to 2%. For cases where the ratio of starting substance and product is desired it is not necessary to add an internal standard.

6.4.7.3 Gas Chromatography

Gas chromatography is sometimes used to study the rates and/or course of a solid state reaction. However, because the method involved both dissolving and heating the sample it has inherent drawbacks. Obviously it cannot be used to study solid-state thermal reactions, since the reaction would occur during analysis in the gas chromatography. Gas chromatography, however, is well suited for studying thermally stable substances and has found use in the study of solid-state photochemical reactions as well as desolvations and solid-state hydrolysis reactions. Gas chromatography is rapid, with a typical analysis requiring 5-30 min, and is sensitive. The sensitivity can be greatly enhanced by using a mass spectrometer as a detector.

A typical analysis proceeds in the following steps:
Step 1. A suitable stationary phase (column) is selected.
Step 2. The optimum column temperature, flow rate, and column length are selected.
Step 3. The best detector is chosen.
Step 4. A number of known samples are analyzed, a calibration curve is constructed, and the unknowns are analyzed.

6.4.7.4 High-Pressure Liquid Chromatography (HPLC)

High-pressure liquid chromatography is probably the most widely used analytical method in the pharmaceutical industry. However, because it is a relatively new method (1965-1970), only a few minutes of its use for the study of solid-state reactions are available.

In some ways, a high-pressure liquid chromatography resembles a gas chromatography in that it has an injector, a column, and a detector. However, in high-pressure liquid chromatography it is not necessary to heat the column or sample, making this technique useful for the analysis of heat sensitive substances. In addition, a wide range of column substances are available, ranging from silica to the so-called reversed-phase columns (which are effectively nonpolar columns). As with gas chromatography, several detectors are available. The variable-wavelength ultraviolet detector is particularly useful for pharmaceuticals and for studying the solid-state reactions of pharmaceuticals, since most pharmaceuticals and their reaction products absorb in the ultraviolet range. In addition, extremely sensitive fluorescence and electrochemical detectors are also available.

A typical analysis by HPLC proceeds in the following manner:
Step 1. Selection of column and detector—these selections are usually based on the physical properties of the reactant and the product.
Step 2. Optimization of flow rate and column length to obtain the best separation.
Step 3. Analysis of known mixtures of reactant and product and construction of a calibration curve.

Thin-layer chromatography (TLC) provides a very simple and efficient method of separation. Only minimal equipment is required for TLC, and very good separations can often be achieved. In general, it is difficult to quantitate TLC, so it is usually used as a method for separation of compounds.

A typical investigation of a solid-state reaction with TLC proceeds as follows:
Step 1. The adsorbent (stationery phase) is selected and plates either purchased or prepared. Usually silica gel or alumina are used.
Step 2. The sample and controls, such as unreacted starting substance, are spotted near the bottom of the plate and developed in several solvents until the best separation is discovered.

This procedure then gives the researcher a good idea of the number of products formed. Based on these preliminary studies, an efficient preparative separation of the products and reactant can often be designed and carried out.

6.5 Generation of Arrays of Solid-Forms

High throughput approaches are used to generate large numbers (greater than 10, more typically greater than 50 or 100, or more preferably 1000 or greater samples) of parallel small-scale crystallizations for a given compound-of-interest. To maximize the diversity of distinct solid-forms generated in this approach, a number of parameters, discussed in detail in section 5.2, can be varied across a larger number of samples.

The preferred system is described in more detail below with references to FIGS. 2A-2C. FIG. 2A is a schematic overview of a high-throughput system for generation and analysis of approximately 25,000 solid-forms of an active component.

FIG. 2A shows the overall system, which consists of a series of integrated modules, or workstations. These modules may be connected directly, through an assembly-line approach, using conveyor belts, or may be indirectly connected by human intervention to move substances between modules. Functionally, the system consists of three main modules: sample generation 10, sample incubation 30, and sample detection 50.

As shown in more detail in FIG. 2B, the sample generation module 10 begins with labeling and identification of each plate 14 (for example, using high speed inkjet labeling 16 and bar-code reading 18). Once labeled, the plates 14 proceeds to the dispensing sub-modules. The first dispensing sub-module 20 is where the compound(s)-of-interest are dispensed into the sample wells or sample tube of the plates. Additional dispensing sub-modules 22a, 22b, 24a, and 24b are employed to add compositional diversity. Note there is a minimum of one dispenser in each of these sub-modules, but there can be as many as is practical. One sub-module 22a can dispense anti-solvent to the sample solution. Another sub-module 22b can dispense additional reagents, such as surfactants, crystalizing aids, etc., in order to enhance crystallization. A critical component of one of the sub-modules 24a or 24b is the ability to dispense sub-microliter amounts of liquid. This nanoliter dispensing can involve the use of inkjet technology (in any of its forms) and is preferably compatible with organic solvents. If desired, after dispensing is complete, the plates can be sealed to prevent solvent evaporation. The sealing mechanism 26 can be a glass plate with an integrated chemically compatible gasket (not shown). This mode of sealing allows optical analysis of each sample site without having to remove the seal.

The sealed plates 28 from the sample generation module next enter into the sample incubation module 30, shown in FIG. 2C. The incubation module 30 consists of four sub-modules. The first sub-module is a heating chamber 32. In one example of use of the incubation chamber, the sample plates can be heated to a temperature (T1). This heating dissolves any compounds that may have undergone precipitation in the previous process. After incubating at this elevated temperature for a period of time, each well (not shown) can be analyzed for the presence of undissolved solids. Wells that contain solids are identified and can be filtered or tracked throughout the process in order to avoid being deemed a “hit” in the final analysis. After the heating treatment, the plates can be subjected to a cooling treatment to a final temperature T2, using, cooling sub-module 34. Preferably, this cooling sub-module 34 maintains uniform temperature across each plate in the chamber (+/−1 decree C.). At this point, if desired, the samples can be subjected to a nucleating event from nucleation station 33. Nucleation events include mechanical stimulation and exposure to sources of energy, such as acoustic (ultrasound), electrical, or laser energy. A nucleation also includes addition of nucleation promoters or other components, such as additives that decrease the surface energy or seed crystals of the compound-of-interest. During cooling, each sample is analyzed for the presence of solid formation. This analysis allows the determination of the temperature at which crystallization or precipitation occurred.

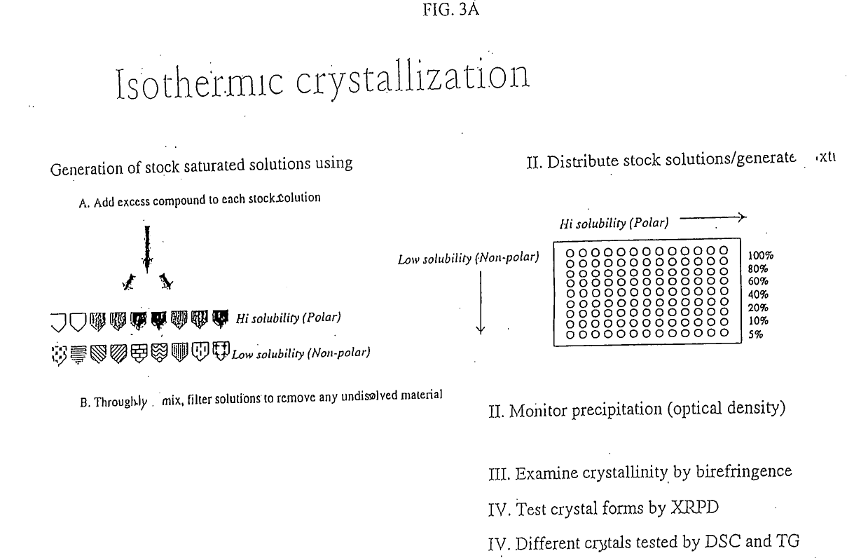

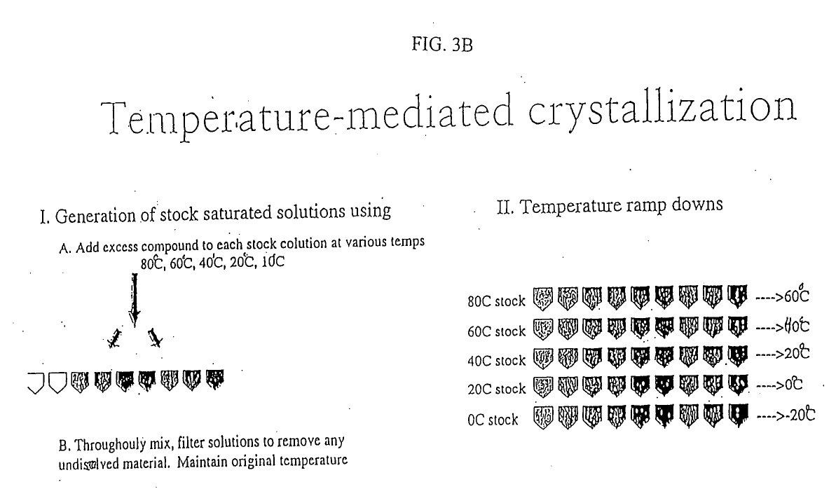

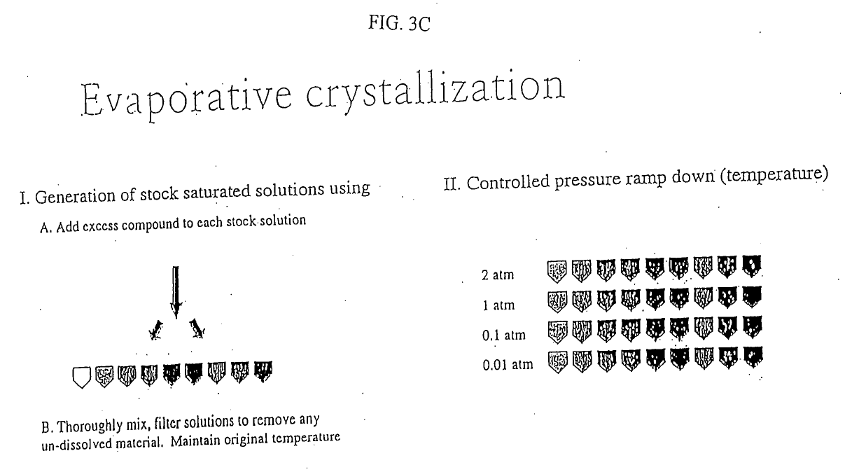

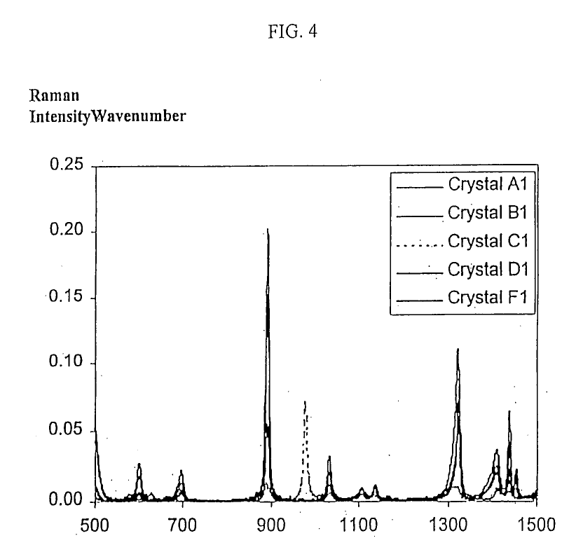

FIGS. 3A-3C are schematics of combinatorial sample processing to produce new polymorphs (on a scale of 10,000 crystallization attempts/pharmaceutical). Three types of crystallization: isothermic, temperature-mediated, and evaporative crystallization, are shown schematically in FIGS. 3A-3C.

Isothemic crystallization of a pharmaceutical as the compound-of-interest is shown in FIG. 3A. Stock saturated solutions are prepared by adding pharmaceutical to solvent in excess of the amount that will go into solution. Then, for example, pharmaceutical is added to a series of different solvents, ranging in polarity from extremely polar to non-polar, and mixtures thereof (from 100% polar to 100% non-polar). The pharmaceutical solutions are mixed, then filtered to remove any undissolved substance. Precipitation is monitored by optical density using standard spectrophotometric methods. Crystallinity is examined by birefringence. Crystal forms are analyzed by XRPD, DSC, melting point (MP) and TG, or other means for thermal analyses.

Temperature mediated crystallization is shown in FIG. 3B. Stock saturated solutions are generated by adding excess compound to each stock solution at various temperatures, for example, 80° C., 60° C., 40° C., 20° C., and 10° C. The solutions are thoroughly mixed, then filtered to remove any undissolved substance while maintaining the original temperature. Temperature is then decreased, each well to a different temperature, for example, the 80° C. stock solution is decreased in nine increments to 60° C., the 60° C. stock solution is decreased in nine increments to 40° C., etc. The resulting samples are then assayed for precipitation, crystallinity, and crystalline forms, as described in FIG. 3A. Evaporative crystallization is shown in FIG. 3C. As in the previous two examples, stock saturated solutions are prepared by adding an excess of pharmaceutical to solvent, mixing, and removing undissolved substance. Temperature is maintained at a constant throughout processing. Pressure can be then decreased, for example, from 2 atmospheres to 1, to 0.1 to 0.01 atm, to generate multiple samples. Referring back to FIG. 2C, after the cooling treatment is complete, the solvent in the wells of the plates is removed, for example, by filtration or evaporation, in order to quench the crystallization process. The solvent removal occurs at the third sub-module 30 of the incubation module.

Other types of crystallization include introducing a precipitation event, such as adding a non-solvent; simply allowing a saturated solution to incubate for a period of time (ageing); or introducing a nucleation event, such as seeding of a saturated solution using one or more crystals of a particular structure. The seed crystal acts as a nucleation site for the formation of the additional crystal structure. An array of crystal forms can be created by using the robotic arm to introduce a single different crystal seed into each well containing the saturated pharmaceutical solution.

6.5.1 Procedure for Analysis of Crystal Forms

Referring back to FIG. 2C, after solvent removal, each well is analyzed for the presence of crystal formation. The analyses are carried out in the fourth sub-module 50.

In the preferred embodiment, this sub-module utilizes machine vision technology Specifically, images are captured by a high-speed charge-coupled device (CCD) camera that has an on-board signal processor. This on-board processor is capable of rapid processing of the digital information contained in the images of the sample tubes or sample wells. Typically, two images are generated for each location of the well that is being analyzed. These two images differ only in that each is generated under different incident light polarization. Differences in these images due to differential rotation of the polarized light indicates the presence of crystals. For wells that contain crystals, the vision system determines the number of crystals in the well, the exact spatial location of the crystals within the well (e.g., X and Y coordinates) and the size of each crystal. This size information, measured as the aspect ratio of the crystal, corresponds to crystal habit. The use of on-line machine vision to determine both the absence/presence of crystals as well as detailed spatial and morphological information has significant advantages. Firstly, this analysis provides a “filtering” means to reduce the number of samples that will ultimately undergo in-depth analysis. This is critical to the functional utility of the system, as in-depth analysis of all samples would be intractable. Additionally, this filtering is achieved with high confidence that the wells analyzed truly contain crystals. Secondly, the spatial information gathered on the locations of crystals is critical to the efficiency in which the in-depth analyses can be performed. This information allows for the specific analysis of individual crystals that are two to four orders of magnitude smaller than the wells in which they are contained.

Those wells (reservoirs or sites in the array) identified to contain crystalline or other specific solid-forms of the compound to be screened are selected for analysis using spectroscopic methods such as IR, NIR or RAMAN spectroscopy as well as XRP Diffractometry. Video optical microscopy and image analysis can be used to identify habit and crystal size. Polarized light analysis, near field scanning optical microscopy, and far field scanning optical microscopy can be used to discern different polymorphs in high-throughput modes. Data collected on a large number of individual crystallizations can be analyzed using informatics protocols to group similar polymorphs, hydrates and solvates. Representatives of each family as well as any orphan crystals can be subjected to thermographic analyses including differential scanning calorimetry (DSC).

Analysis of solid-forms for crystal habit can be performed using image-analysis techniques, such as microscopy, photomicrography, electron microscopy, near field scanning optical microscopy, far field scanning optical microscopy, atomic-force microscopy. Analysis concerning polymorphic form can be performed by Raman spectroscopy or XRD. The solid-forms can then screened for solubility, dissolution, and stability. Additional means for analysis include pH sensors, ionic strength sensors, mass spectrometers, optical spectrometers, devices for measuring turbidity, calorimeters, infrared and ultraviolet spectrometers, polarimeters, radioactivity counters, devices measuring conductivity, and heat of dissolution.

The collected data can be analyzed using informatics. Informatics protocols enable high-throughput analysis of spectroscopic, diffractometric, and thermal analyses and thereby enable identification of crystal forms that belong to the same polymorph family. These informatics tools facilitate identification of conditions that define occurrence domains (i.e., thermodynamic and kinetic parameters) that will give rise to a specific crystal form.

The samples are then categorized. For example, the samples can be grouped into:
a. wells containing no precipitate;
b. wells with single polymorph;
c. wells with polymorph mixture;
d. wells with amorphous forms of pharmaceutical; and
e. wells with mixtures of categories b-d.
   If desired, selected samples can be prepared and analyzed on a larger scale, for example, by taking a given mass and seeing how much goes into solution in a given time. Crystals are selected for further analysis using XRPD, DSC, and TG.

6.6 Arrays of Solid-Forms for Identifying Solid-Forms with Advantageous Properties

In one embodiment of the methods discussed herein, a goal is to discover and/or identify solid-forms with the most desirable properties. Representative properties include chemical and/or physical stability of compounds, such as pharmaceuticals and/or pharmaceutical formulations during manufacturing, packaging, distribution, storage and administration (as it relates to the compound-of-interest as well as to the formulation as a whole, and components thereof), pharmaceutical uptake from the gastrointestinal tract or mucosa or other route of administration, pharmaceutical half-life after administration to a patient, pharmaceutical properties, delivery kinetics, and other factors which determine the efficacy and economics of a pharmaceutical. As referred to herein, “stability” includes chemical stability and resistance of a solid phase to a change in form such as a phase change or polymorphic transition. In some cases the pharmaceutical may have a single property that negatively affects uptake, such as hydrophobicity or low solubility. In other cases, it can be a combination of properties. Accordingly, the screening process will typically vary at least one component of the sample and/or one processing parameter, and more typically, multiple components of the formulation and/or multiple processing parameters, and select based on one or more properties of the solid-form as a whole.

The method is useful to crystallize a compound that has evaded crystallization, such as CILISTATIN™, or define additional polymorphs for monomorphic compounds such as aspirin. The method can also be used to reveal additional polymorphs for known polymorphic compounds such as. chloramphenicol, methyl prednisolone or barbital, or to affect distribution of polymorphs in a pharmaceutical of known crystal polymorphism.

For example, if the original compound-of-interest is a pharmaceutical characterized by poor oral uptake, the solubility of a number of crystal forms, prepared by seeding, re-crystallizing the pharmaceutical in a range of salt concentrations, pHs, carriers, or pharmaceutical concentrations, can be simultaneously prepared and tested. Solubility is easily examined, for example, by measuring optical density of polymorph dissolved at a known concentration in a solvent such as buffered water, or by measuring the optical density of sample filtrate, pulled through the filter at the bottom of an array using vacuum, where undissolved pharmaceutical remains in the wells of the array. Once “true polymorphs” are identified, then the samples are tested for additional properties such as dissolution (for example, in water), solubility, absorbance (optional, specific to pharmaceutical), and stability.

An ideal crystal or other solid-form of a compound can be defined depending on the particular endpoint application of the compound. These endpoints include pharmaceutical uptake and delivery, dissolution, solid state chemical stability, pharmaceutical processing and manufacture, behavior in suspensions, optical properties, aerodynamic properties, electrical properties, acoustical properties, coating, and co-crystallization with other compounds. For example, the crystal habit of a particular compound will influence the overall shape, size, and mass of particles derived from that substance. This in turn will influence other properties, such as the aerodynamic properties as they relate to pulmonary pharmaceutical delivery. The extent that the particles become separated from each other, their ability to become suspended in air and their ability to fall out of suspension and become deposited in the proper location of the human airways are properties that are all influenced ultimately the crystal form. The ideal crystal form in this case would be the form that optimizes the ability of the substance to achieve optimal airways pharmaceutical delivery using the appropriate medical device (inhaler). In a similar manner, the ideal crystal form can be defined for each of the other endpoints listed above. The best powder flow characteristics are achieved by equiaxed crystals that are tens of micron sized. High surface area crystals have the highest dissolution rates.

In a preferred embodiment, to select optimal crystal forms for oral delivery of a pharmaceutical, a system designed using the disclosure herein, assays crystal forms based on physical parameters, such as absorption, bioavailability, permeability, or metabolism, all using simple, rapid, in vitro testing. In the most preferred embodiment, the various crystal forms are first screened for solubility by measuring the rate of dissolution of each sample. Solubility can be measured using standard technology such as optical density or by colorimetry. Those candidates that look promising are then screened for permeability-passage into the gastrointestinal tract—using a system such as an Ussing chamber. Absorption can be measured using an in vitro assay such as an Ussing chamber containing HT Caco-2/MS engineered cells (Lennemas, H, J. Pharm. Sci. 87(4), 403-410, April 1998). As used in this context, permeability generally refers to the permeability of the intestinal wall with respect to the pharmaceutical, i.e., how much pharmaceutical gets through. Metabolism of the compounds are then tested using in vitro assays. Metabolism can be measured using digestive enzymes and cell lines, such as hepatoma cell lines which are indicative of the effect of the liver on pharmaceutical metabolism.

In vitro screening, as used herein, includes testings for any number of physiological or biological activities, whether known or later recognized. The new crystal forms can be screened for the known activity of the pharmaceutical. Alternatively, since a change in crystal form can also change bioactivity, each pharmaceutical crystal form can also or alternatively be subjected to a battery of in vitro screening tests for multiple activities, such as antibacterial activity, antiviral activity, antifungal activity, antiparasitic activity, cytotherapeutic activity (especially against one or more types of cancer or tumor cells), alteration of metabolic function of eukaryotic cells, binding to specific receptors, modulation of inflammation and/or immunomodulation, modulation of angiogenesis, anticholinergic activity, and modulation of enzyme levels or activity. Metabolic function testing includes sugar metabolism, cholesterol uptake, lipid metabolism, and blood pressure regulation, amino acid metabolism, nucleoside/nucleotide metabolism, amyloid formation, and dopamine regulation. Compounds can also be screened for delivery parameters, for example, for pulmonary delivery it is desirable to look at aerodynamic parameters including conformation, total surface area, and density.

These screening tests include any that are presently known, and those that are later developed. Typically the initial screening test is an in vitro assay that is routinely used in the field. The preferred assays yield highly reliable and reproducible results, can be performed quickly, and give results predictive of in vivo results. Numerous in vitro screening tests are known. For example, receptor binding assays as a primary pharmaceutical screen is discussed in Creese, I. Neurotransmitter Receptor Binding, pp. 189-233 (Yamamura, et al, editors) (2d ed. 1985). Another example is an assay for detecting cytotherapeutic activity against cancer.

After in vitro screening, the crystal forms that have been identified as having optimal characteristics will undergo testing in one or more animal or tissue models and ultimately, in humans. Safety is evaluated in animals by LD50 measurements and other toxicologic methods of evaluation (liver function tests, hematocrit, etc.). Efficacy is evaluated in specific animal models for the type of problem for which treatment is sought.

6.7 Arrays to Identify Conditions and Additives for Enantiomeric Resolution of Racemates by Direct Crystallization

Chiral compounds that can exist as crystalline conglomerates can be enantiomerically resolved by crystallization. Conglomerate behavior means that under certain crystallization conditions, optically-pure, discrete crystals or crystal clusters of both enantiomers will form, although, in bulk, the conglomerate is optically neutral. Racemic chiral compounds that display conglomerate behavior can be enantiomerically resolved by preferential crystallization (i.e., crystallizing one enantiomer from a supersaturated solution of a racemate, for example, by seeding the solution with the pure enantiomer). Of course, before preferential crystallization can be employed, it is necessary to establish that the compound exhibits conglomerate behavior. For this, one may utilize the invention described herein for high-throughput screening to find suitable conditions, such as time, temperature, solvent mixtures, and additives, etc. that result in a conglomerate. Well-known properties for which compounds can be tested to determine if they are potential conglomerates include: (1) melting point (if the melting point of one enantiomer exceeds that of the racemate by 25° C. or more, the probability that the compound can form a conglomerate is high); (2) demonstration of spontaneous resolution via measurement of a finite optical rotation of a solution prepared from a single crystal, x-ray analysis of a single crystal, or solid-state IR analysis of a single crystal compared with the spectrum of the racemate (if the solid-state IR of the single crystal and that of the racemate are identical, there is a high probability that the compound is a conglomerate); or (3) solubility behavior of one of the enantiomers in a saturated solution of the racemate. Insolubility is indicative of conglomerate behavior. Eliel et al., Stereochemistry of Organic Compounds, John Wiley & Sons, Inc. New York (1994), p. 301, incorporated herein by reference. Thus an array can be prepared to determine conglomerate behavior of a particular compound-of-interest by preparing samples containing the compound-of-interest and various components, solvents, and solvent mixtures. For example, the array can be prepared by varying solvents, solvent mixtures, and solvent concentrations between samples, the object find the particular solvent system(s) that give the best results. Preferably, one or more of the samples differs from one or more other samples by:
(a) the amount or the concentration of the compound-of-interest;
(b) the identity of one or more of the components;
(c) the amount or the concentration of one or more of the components;
(d) the physical state of one or more of the components; or
(e) the value of pH.

For example, samples can have one or more of the following components at various concentrations: excipients; solvents; salts; acids; bases; gases; small molecules, such as hormones, steroids, nucleotides, nucleosides, and aminoacids; large molecules, such as oligonucleotides, polynucleotides, oligonucleotide and polynucleotide conjugates, proteins, peptides, peptidomimetics, and polysaccharides; pharmaceuticals; dietary supplements; alternative medicines; nutraceuticals; sensory compounds; agrochemicals; the active component of a consumer formulation; and the active component of an industrial formulation; crystallization additives, such as additives that promote and/or control nucleation, additives that affect crystal habit, and additives that affect polymorphic form; additives that affect particle or crystal size; additives that structurally stabilize crystalline or amorphous solid-forms; additives that dissolve solid-forms; and additives that inhibit crystallization or solid formation; optically-active solvents; optically-active reagents; and optically-active catalysts.

The array is then processed according to the objective of the experiment, for example, by adjusting the value of the temperature; adjusting the time of incubation; adjusting the pH; adjusting the amount or the concentration of the compound-of-interest; adjusting the amount or the concentration of one or more of the components; adding one or more additional components; nucleation (e.g., an optically pure seed crystal to induce preferential crystallization); or controlling the evaporation of one or more of the components, such as the solvent (e.g., adjusting a value of pressure or adjusting the evaporative surface area); or a combination thereof.

After processing according to the methods described in Section 4.5 above, the samples can be analyzed as described in Section 6.4, first to identify those samples with crystals then to identify those crystals exhibiting conglomerate behavior, e.g., formation of individual enantiomerically-pure crystal aggregates. Preferably, analysis is performed using on-line automated equipment. For example, the samples can be filtered and solid-state IR analysis or x-ray-powder-diffraction studies can be preformed on the filtered material. Alternatively, optical-rotation studies can be performed on the filtrate in cases where an optically-pure seed crystal was added to induce preferential crystallization.

6.8 Arrays to Identify Conditions for Resolution of Enantiomers Via Diastereomers

Enantiomeric resolution of a racemic mixture of a chiral compound can be effected by: (1) conversion into a diastereomeric pair by treatment with an enantiomerically-pure chiral substance, (2) preferential crystallization of one diastereomer over the other, followed by (3) conversion of the resolved diastereomer into the optically-active-enantiomer. Neutral compounds can be converted in diastereomeric pairs by direct synthesis or by forming inclusions, while acidic and basic compounds can be converted into diastereomeric salts. Finding suitable diastereomeric-pair-forming reagents and crystallization conditions can involve testing hundreds of reagents that can form salts, reaction products, charge transfer complexes, or inclusions with the compound-of-interest. Such testing can be conveniently accomplished using the high-throughput arrays and methods disclosed herein. Thus, each sample in an array of the invention can be a miniature reaction vessel, each comprising a reaction between the compound-of-interest and an optically-pure compound. Samples are then analyzed for solid formation and whether formation and/or preferential crystallization of one diastereomer of a diastereomeric pair occurred. Once potential diastereomeric pairs are discovered, the invention provides methods to test a large number of components, solvents, and conditions to find optimal conditions for preferential crystallization of one diastereomer of the diastereomeric pair. For example, the array can be prepared by varying solvents, solvent mixtures, and solvent concentrations between samples, the object find the particular solvent system(s) that give the best results. Preferably, one or more of the samples differs from one or more other samples by:
(a) the amount or the concentration of the diastereomeric derivative of the compound-of-interest;
(b) the identity of the diastereomeric derivative of the compound-of-interest;
(c) the identity of one or more of the components;
(d) the amount or the concentration of one or more of the components;
(e) the physical state of one or more of the components; or
(f) the value of pH.

For example, samples can have one or more of the following components at various concentrations: excipients; solvents; salts; acids; bases; gases; small molecules, such as hormones, steroids, nucleotides, nucleosides, and aminoacids; large molecules, such as oligonucleotides, polynucleotides, oligonucleotide and polynucleotide conjugates, proteins, peptides, peptidomimetics, and polysaccharides; pharmaceuticals; dietary supplements; alternative medicines; nutraceuticals; sensory compounds; agrochemicals; the active component of a consumer formulation; and the active component of an industrial formulation; crystallization additives, such as additives that promote and/or control nucleation, additives that affect crystal habit, and additives that affect polymorphic form; additives that affect particle or crystal size; additives that structurally stabilize crystalline or amorphous solid-forms; additives that dissolve solid-forms; and additives that inhibit crystallization or solid formation; optically-active solvents; optically-active reagents; and optically-active catalysts.

The array is then processed as discussed in Section 4.5 above, according to the objective of the experiment, for example, by adjusting the value of the temperature; adjusting the time of incubation; adjusting the pH; adjusting the amount or the concentration of the compound-of-interest; adjusting the amount or the concentration of one or more of the components; adding one or more additional components; nucleation (e.g., an optically pure seed crystal to induce preferential crystallization); or controlling the evaporation of one or more of the components, such as the solvent (e.g., adjusting a value of pressure or adjusting the evaporative surface area); or a combination thereof.

After processing, the samples can be analyzed, as described in Section 6.4, first to identify those samples with crystals, the crystals can be further analyzed by well-known methods to determine if they are diastereomerically-enriched. Preferably, analysis is performed using on-line automated equipment. For example, the samples can be filtered and analytical methods such as HPLC, gas chromatography, and liquid chromatography-mass spectroscopy (LC-MS) can be performed to determine diastereomeric purity. Alternatively, the diastereomer can be converted back to the enantiomer by well-known methods depending on its identity and optical-activity analysis performed, such as chiral-phase HPLC, chiral-phase gas chromatography, chiral-phase liquid chromatography/mass spectroscopy (LC-MS), and optical-rotation measurement.

6.9 Arrays to Identify Conditions, Compounds, or Compositions that Prevent or Inhibit Crystallization, Precipitation, Formation, or Deposition of Solid-Forms

In a separate embodiment, the invention is useful to discover or optimize conditions, compounds, or compositions that prevent or inhibit crystallization, precipitation, formation, or deposition of solid-forms. For example, an array can be prepared comprising samples having the appropriate medium (combination of components, preferably, including a solvent as one of the components) and having a dissolved compound-of-interest. The array is then processed. If desired, particular samples can be processed under various conditions including, but not limited to, adjusting the temperature; adjusting the time; adjusting the pH; adjusting the amount or the concentration of the compound-of-interest; adjusting the amount or the concentration of a component; component identity (adding one or more additional components); adjusting the solvent removal rate; introducing of a nucleation event; introducing of a precipitation event; controlling evaporation of the solvent (e.g., adjusting a value of pressure or adjusting the evaporative surface area); or adjusting the solvent composition, or a combination thereof. Preferably, one or more of the samples differs from one or more other samples by:
(a) the amount or the concentration of the compound-of-interest;
(b) the identity of one or more of the components;
(c) the amount or the concentration of one or more of the components;
(d) a physical state of one or more of the components; or
(e) pH.

For example, samples can have one or more of the following components at various concentrations: excipients; solvents; salts; acids; bases; gases; small molecules, such as hormones, steroids, nucleotides, nucleosides, and aminoacids; large molecules, such as oligonucleotides, polynucleotides, oligonucleotide and polynucleotide conjugates, proteins, peptides, peptidomimetics, and polysaccharides; pharmaceuticals; dietary supplements; alternative medicines; nutraceuticals; sensory compounds; agrochemicals; the active component of a consumer formulation; and the active component of an industrial formulation; crystallization additives, such as additives that promote and/or control nucleation, additives that affect crystal habit, and additives that affect polymorphic form; additives that affect particle or crystal size; additives that structurally stabilize crystalline or amorphous solid-forms; additives that dissolve solid-forms; and additives that inhibit crystallization or solid formation; optically-active solvents; or optically-active reagents.

After processing, according to the disclosure presented in Section 4.5, the samples can be analyzed, according to the methods discussed in Section 6.4, to identify those samples having a solid-form and those that do not. The samples that do not have solid-forms are predicative of conditions, compounds, or compositions that prevent or inhibit crystallization, precipitation, formation, or deposition of solid-forms. The positive samples can be further analyzed to determine the solid-form's structural, physical, pharmacological, or chemical properties.

6.10 Arrays to Identify Conditions, Compounds, or Compositions that Promote Dissolution, Destruction, or Breakup of Solid-Forms

In another embodiment, the invention is useful to discover or optimize conditions, compounds, and compositions that promote dissolution, destruction, or breakup of inorganic and organic solid-forms. In this embodiment, an array is prepared comprising samples having the appropriate medium and having a solid-form of the compound-of-interest. Then, if desired, various components in varying concentrations can be added to selected samples and the samples processed. Particular samples can be processed under various conditions. Preferably, one or more of the samples differs from one or more other samples by:
(a) the amount or the concentration of the compound-of-interest;
(b) the physical state the compound-of-interest;
(c) the identity of one or more of the components;
(d) the amount or the concentration of one or more of the components;
(e) a physical state of one or more of the components; or
(f) pH.

For example, samples can have one or more of the following components at various concentrations: excipients; solvents; salts; acids; bases; gases; small molecules, such as hormones, steroids, nucleotides, nucleosides, and aminoacids; large molecules, such as oligonucleotides, polynucleotides, oligonucleotide and polynucleotide conjugates, proteins, peptides, peptidomimetics, and polysaccharides; pharmaceuticals; dietary supplements; alternative medicines; nutraceuticals; sensory compounds; agrochemicals; the active component of a consumer formulation; and the active component of an industrial formulation; crystallization additives, such as additives that promote and/or control nucleation, additives that affect crystal habit, and additives that affect polymorphic form; additives that affect particle or crystal size; additives that structurally stabilize crystalline or amorphous solid-forms; additives that dissolve solid-forms; additives that inhibit crystallization or solid formation; optically-active solvents; and optically-active reagents.

After processing, according to the disclosure presented in Section 4.5, the samples can be analyzed, according to the methods discussed in Section 6.4, to identify positive samples, i.e., samples wherein the solid-form of the compound-of-interest chanced in physical state, such as by partially or fully dissolving, by fragmenting, by increasing surface-to-volume ratio, by polymorphic shift, by change in crystal habit, or has otherwise been rendered physically, structurally, or chemically different. Thus, one or more of the compound-of-interest's structural, physical, pharmacological, or chemical properties can be measured or determined.

### 7. EXAMPLE

The following Example further illustrate the method and arrays of the present invention. It is to be understood that the present invention is not limited to the specific details of the Example provided below.

7.1 Preparation and Identification of Glycine Crystals

A stock solution of alycine was prepared by dissolving 240 g of glycine in one liter of deionized water. An appropriate amount (278 μl) of this stock solution was deposited, in individual 0.75 ml glass vials arranged in an 8×12 array (total number of vials is 96). Labels were assigned to each vial according to position in the array, where columns were described by a number 1 through 12 and rows a letter A through H. The solvent was removed via evaporation under vacuum to yield solid glycine in each vial. To each vial, 200 microliters of the solvent was added. Chosen solvents were aqueous solutions of varying pH, where the pH of each solution was adjusted using acetic acid, sulfuric acid, and/or ammonium hydroxide. crystallization additives were chosen from a library consisting of α-amino acids as either pure enantiomers or racenic mixtures and ampiphilic. Selected crystallization additives included DL-alanine, DL-serine, L-threonine, L-phenylalanine and Triton X-100. All crystallization additives were supplied by Sigma Chemicals, Inc. The concentration of crystallization additives was either 0.1 or 1 0.0 wt % based on the dry weight of glycine. Table 6.1 gives the specific composition of each vial of such a 96. vial array. The formulated sample vials were heated at 80.0° C. for approximately 30 minutes in a temperature controlled heating/cooling block to dissolve the glycine. Upon complete dissolution of the glycine. the samples were cooled to room temperature (25° C.) at a rate of 1° C. per minute, yielding crystals of varying form/habit. Crystals were harvested from individual vials by decanting off the supernatant and characterized using single crystal laser Raman spectroscopy and digital optical microscopy.

7.2 Results

The content of each well of the 96 vial array are summarized in Table 6.2. The laser Raman spectra of representative; randomly oriented glycine crystals were measured at room temperature using a Bruker FT Raman Spectrometer, model RES 100/S (Bruker Optics, Inc.). The Raman intensity is plotted as a function of wavenumber in Figure 6.1 for representative samples. The spectra obtained for samples A1, B1, D1 and F1 can be matched to the spectra for standard glycine. The appearance of new Raman peaks, for example, at wavenumbers of 863 and 975, in sample C1 indicates a difference in crystal structure relative to crystals A1, B1, D1, and F1, suggesting a different polymorphic structure for crystal C1. Different crystal habits were observed for crystals grown from different formulations. These results demonstrate the ability to tailor crystal habit by controlling crystallization formulation as shown in Table 6.1 and 6.2 below.

TABLE 6.1
Formulation in Individual Vials of the 96 vial array.
(v/o stands for percent volume)

| | | | | | | crystallization | | |
|---|---|---|---|---|---|---|---|---|
| | | glycine | super- | | | additive | | |
| Vial | | concentration | saturation | | crystallization | concentration | wt crystallization | |
| # | g glycine | (g/ml) | (%) | Solvent | additive | (wt %) | additive | μl solvent |
| A1 | 0.06672 | 0.3336 | 32.9 | deionized water | none | 0 | 0 | 200 |
| A2 | 0.06672 | 0.3336 | 32.9 | deionized water | none | 0 | 0 | 200 |
| A3 | 0.06672 | 0.3336 | 32.9 | deionized water | none | 0 | 0 | 200 |
| A4 | 0.06672 | 0.3336 | 32.9 | deionized water | none | 0 | 0 | 200 |
| A5 | 0.06672 | 0.3336 | 32.9 | deionized water | none | 0 | 0 | 200 |
| A6 | 0.06672 | 0.3336 | 32.9 | deionized water | none | 0 | 0 | 200 |
| A7 | 0.06672 | 0.3336 | 32.9 | deionized water | none | 0 | 0 | 200 |
| A8 | 0.06672 | 0.3336 | 32.9 | deionized water | none | 0 | 0 | 200 |
| A9 | 0.06672 | 0.3336 | 32.9 | deionized water | none | 0 | 0 | 200 |
| A10 | 0.06672 | 0.3336 | 32.9 | deionized water | none | 0 | 0 | 200 |
| A11 | 0.06672 | 0.3336 | 32.9 | deionized water | none | 0 | 0 | 200 |
| A12 | 0.06672 | 0.3336 | 32.9 | deionized water | none | 0 | 0 | 200 |
| B1 | 0.06672 | 0.3336 | 32.9 | 4 v/o acetic acid | none | 0 | 0 | 200 |
| | | | | solution | | | | |
| B2 | 0.06672 | 0.3336 | 32.9 | 4 v/o acetic acid | none | 0 | 0 | 200 |
| | | | | solution | | | | |
| B3 | 0.06672 | 0.3336 | 32.9 | 4 v/o acetic acid | none | 0 | 0 | 200 |
| | | | | solution | | | | |
| B4 | 0.06672 | 0.3336 | 32.9 | 4 v/o acetic acid | none | 0 | 0 | 200 |
| | | | | solution | | | | |
| B5 | 0.06672 | 0.3336 | 32.9 | 4 v/o acetic acid | none | 0 | 0 | 200 |
| | | | | solution | | | | |
| B6 | 0.06672 | 0.3336 | 32.9 | 4 v/o acetic acid | none | 0 | 0 | 200 |
| | | | | solution | | | | |
| B7 | 0.06672 | 0.3336 | 32.9 | 4 v/o acetic acid | none | 0 | 0 | 200 |
| | | | | solution | | | | |
| B8 | 0.06672 | 0.3336 | 32.9 | 4 v/o acetic acid | none | 0 | 0 | 200 |
| | | | | solution | | | | |
| B9 | 0.06672 | 0.3336 | 32.9 | 4 v/o acetic acid | none | 0 | 0 | 200 |
| | | | | solution | | | | |
| B10 | 0.06672 | 0.3336 | 32.9 | 4 v/o acetic acid | none | 0 | 0 | 200 |
| | | | | solution | | | | |
| B11 | 0.06672 | 0.3336 | 32.9 | 4 v/o acetic acid | none | 0 | 0 | 200 |
| | | | | solution | | | | |
| B12 | 0.06672 | 0.3336 | 32.9 | 4 v/o acetic acid | none | 0 | 0 | 200 |
| | | | | solution | | | | |
| C1 | 0.06672 | 0.3336 | 32.9 | 6 v/o sulfuric | none | 0 | 0 | 200 |
| | | | | acid solution | | | | |
| C2 | 0.06672 | 0.3336 | 32.9 | 6 v/o sulfuric | none | 0 | 0 | 200 |
| | | | | acid solution | | | | |
| C3 | 0.06672 | 0.3336 | 32.9 | 6 v/o sulfuric | none | 0 | 0 | 200 |
| | | | | acid solution | | | | |
| C4 | 0.06672 | 0.3336 | 32.9 | 6 v/o sulfuric | none | 0 | 0 | 200 |
| | | | | acid solution | | | | |
| C5 | 0.06672 | 0.3336 | 32.9 | 6 v/o sulfuric | none | 0 | 0 | 200 |
| | | | | acid solution | | | | |
| C6 | 0.06672 | 0.3336 | 32.9 | 6 v/o sulfuric | none | 0 | 0 | 200 |
| | | | | acid solution | | | | |
| C7 | 0.06672 | 0.3336 | 32.9 | 6 v/o sulfuric | none | 0 | 0 | 200 |
| | | | | acid solution | | | | |
| C8 | 0.06672 | 0.3336 | 32.9 | 6 v/o sulfuric | none | 0 | 0 | 200 |
| | | | | acid solution | | | | |
| C9 | 0.06672 | 0.3336 | 32.9 | 6 v/o sulfuric | none | 0 | 0 | 200 |
| | | | | acid solution | | | | |
| C10 | 0.06672 | 0.3336 | 32.9 | 6 v/o sulfuric | none | 0 | 0 | 200 |
| | | | | acid solution | | | | |
| C11 | 0.06672 | 0.3336 | 32.9 | 6 v/o sulfuric | none | 0 | 0 | 200 |
| | | | | acid solution | | | | |
| C12 | 0.06672 | 0.3336 | 32.9 | 6 v/o sulfuric | none | 0 | 0 | 200 |
| | | | | acid solution | | | | |
| D1 | 0.06672 | 0.3336 | 32.9 | deionized water | Triton X-100 | 0.10 | 0.006672 | 200 |
| D2 | 0.06672 | 0.3336 | 32.9 | deionized water | Triton X-100 | 0.10 | 0.006672 | 200 |
| D3 | 0.06672 | 0.3336 | 32.9 | deionized water | Triton X-100 | 0.10 | 0.006672 | 200 |
| D4 | 0.06672 | 0.3336 | 32.9 | deionized water | Triton X-100 | 0.10 | 0.006672 | 200 |
| D5 | 0.06672 | 0.3336 | 32.9 | deionized water | Triton X-100 | 0.10 | 0.006672 | 200 |
| D6 | 0.06672 | 0.3336 | 32.9 | deionized water | Triton X-100 | 0.10 | 0.006672 | 200 |
| D7 | 0.06672 | 0.3336 | 32.9 | deionized water | Triton X-100 | 10.00 | 0.6672 | 200 |
| D8 | 0.06672 | 0.3336 | 32.9 | deionized water | Triton X-100 | 10.00 | 0.6672 | 200 |
| D9 | 0.06672 | 0.3336 | 32.9 | deionized water | Triton X-100 | 10.00 | 0.6672 | 200 |
| D10 | 0.06672 | 0.3336 | 32.9 | deionized water | Triton X-100 | 10.00 | 0.6672 | 200 |
| D11 | 0.06672 | 0.3336 | 32.9 | deionized water | Triton X-100 | 10.00 | 0.6672 | 200 |
| D12 | 0.06672 | 0.3336 | 32.9 | deionized water | Triton X-100 | 10.00 | 0.6672 | 200 |
| E1 | 0.06672 | 0.3336 | 32.9 | deionized water | DL-alanine | 0.10 | 0.006672 | 200 |
| E2 | 0.06672 | 0.3336 | 32.9 | deionized water | DL-alanine | 0.10 | 0.006672 | 200 |
| E3 | 0.06672 | 0.3336 | 32.9 | deionized water | DL-alanine | 0.10 | 0.006672 | 200 |
| E4 | 0.06672 | 0.3336 | 32.9 | deionized water | DL-alanine | 0.10 | 0.006672 | 200 |
| E5 | 0.06672 | 0.3336 | 32.9 | deionized water | DL-alanine | 0.10 | 0.006672 | 200 |
| E6 | 0.06672 | 0.3336 | 32.9 | deionized water | DL-alanine | 0.10 | 0.006672 | 200 |
| E7 | 0.06672 | 0.3336 | 32.9 | deionized water | DL-alanine | 10.00 | 0.6672 | 200 |
| E8 | 0.06672 | 0.3336 | 32.9 | deionized water | DL-alanine | 10.00 | 0.6672 | 200 |
| E9 | 0.06672 | 0.3336 | 32.9 | deionized water | DL-alanine | 10.00 | 0.6672 | 200 |
| E10 | 0.06672 | 0.3336 | 32.9 | deionized water | DL-alanine | 10.00 | 0.6672 | 200 |
| E11 | 0.06672 | 0.3336 | 32.9 | deionized water | DL-alanine | 10.00 | 0.6672 | 200 |
| E12 | 0.06672 | 0.3336 | 32.9 | deionized water | DL-alanine | 10.00 | 0.6672 | 200 |
| F1 | 0.06672 | 0.3336 | 32.9 | deionized water | DL-serine | 0.10 | 0.006672 | 200 |
| F2 | 0.06672 | 0.3336 | 32.9 | deionized water | DL-Serine | 0.10 | 0.006672 | 200 |
| F3 | 0.06672 | 0.3336 | 32.9 | deionized water | DL-serine | 0.10 | 0.006672 | 200 |
| F4 | 0.06672 | 0.3336 | 32.9 | deionized water | DL-serine | 0.10 | 0.006672 | 200 |
| F5 | 0.06672 | 0.3336 | 32.9 | deionized water | DL-serine | 0.10 | 0.006672 | 200 |
| F6 | 0.06672 | 0.3336 | 32.9 | deionized water | DL-serine | 0.10 | 0.006672 | 200 |
| F7 | 0.06672 | 0.3336 | 32.9 | deionized water | DL-serine | 10.00 | 0.6672 | 200 |
| F8 | 0.06672 | 0.3336 | 32.9 | deionized water | DL-serine | 10.00 | 0.6672 | 200 |
| F9 | 0.06672 | 0.3336 | 32.9 | deionized water | DL-serine | 10.00 | 0.6672 | 200 |
| F10 | 0.06672 | 0.3336 | 32.9 | deionized water | DL-serine | 10.00 | 0.6672 | 200 |
| F11 | 0.06672 | 0.3336 | 32.9 | deionized water | DL-serine | 10.00 | 0.6672 | 200 |
| F12 | 0.06672 | 0.3336 | 32.9 | deionized water | DL-serine | 10.00 | 0.6672 | 200 |
| G1 | 0.06672 | 0.3336 | 32.9 | deionized water | L-threonine | 0.10 | 0.006672 | 200 |
| G2 | 0.06672 | 0.3336 | 32.9 | deionized water | L-threonine | 0.10 | 0.006672 | 200 |
| G3 | 0.06672 | 0.3336 | 32.9 | deionized water | L-threonine | 0.10 | 0.006672 | 200 |
| G4 | 0.06672 | 0.3336 | 32.9 | deionized water | L-threonine | 0.10 | 0.006672 | 200 |
| G5 | 0.06672 | 0.3336 | 32.9 | deionized water | L-threonine | 0.10 | 0.006672 | 200 |
| G6 | 0.06672 | 0.3336 | 32.9 | deionized water | L-threonine | 0.10 | 0.006672 | 200 |
| G7 | 0.06672 | 0.3336 | 32.9 | deionized water | L-threonine | 10.00 | 0.6672 | 200 |
| G8 | 0.06672 | 0.3336 | 32.9 | deionized water | L-threonine | 10.00 | 0.6672 | 200 |
| G9 | 0.06672 | 0.3336 | 32.9 | deionized water | L-threonine | 10.00 | 0.6672 | 200 |
| G10 | 0.06672 | 0.3336 | 32.9 | deionized water | L-threonine | 10.00 | 0.6672 | 200 |
| G11 | 0.06672 | 0.3336 | 32.9 | deionized water | L-threonine | 10.00 | 0.6672 | 200 |
| G12 | 0.06672 | 0.3336 | 32.9 | deionized water | L-threonine | 10.00 | 0.6672 | 200 |
| H1 | 0.06672 | 0.3336 | 32.9 | deionized water | L-phenylalanine | 0.10 | 0.006672 | 200 |
| H2 | 0.06672 | 0.3336 | 32.9 | deionized water | L-phenylalanine | 0.10 | 0.006672 | 200 |
| H3 | 0.06672 | 0.3336 | 32.9 | deionized water | L-phenylalanine | 0.10 | 0.006672 | 200 |
| H4 | 0.06672 | 0.3336 | 32.9 | deionized water | L-phenylalanine | 0.10 | 0.006672 | 200 |
| H5 | 0.06672 | 0.3336 | 32.9 | deionized water | L-phenylalanine | 0.10 | 0.006672 | 200 |
| H6 | 0.06672 | 0.3336 | 32.9 | deionized water | L-phenylalanine | 0.10 | 0.006672 | 200 |
| H7 | 0.06672 | 0.3336 | 32.9 | deionized water | L-phenylalanine | 10.00 | 0.6672 | 200 |
| H8 | 0.06672 | 0.3336 | 32.9 | deionized water | L-phenylalanine | 10.00 | 0.6672 | 200 |
| H9 | 0.06672 | 0.3336 | 32.9 | deionized water | L-phenylalanine | 10.00 | 0.6672 | 200 |
| H10 | 0.06672 | 0.3336 | 32.9 | deionized water | L-phenylalanine | 10.00 | 0.6672 | 200 |
| H11 | 0.06672 | 0.3336 | 32.9 | deionized water | L-phenylalanine | 10.00 | 0.6672 | 200 |
| H12 | 0.06672 | 0.3336 | 32.9 | deionized water | L-phenylalanine | 10.00 | 0.6672 | 200 |

TABLE 6.2
Summary of final content of sample vials.

| | Description of | Relative population of | | | |
|---|---|---|---|---|---|
| Vial # | solid phase | crystals | Crystal Color | Crystal habit | Supernatant color |
| A1 | crystalline | low (<5 crystals) | white/translucent | bipyramidal | clear |
| A2 | crystalline | low (<5 crystals) | white/translucent | bipyramidal | clear |
| A3 | crystalline | low (<5 crystals) | white/translucent | bipyramidal | clear |
| A4 | crystalline | low (<5 crystals) | white/translucent | bipyramidal | clear |
| A5 | crystalline | low (<5 crystals) | white/translucent | bipyramidal | clear |
| A6 | crystalline | low (<5 crystals) | white/translucent | bipyramidal | clear |
| A7 | crystalline | low (<5 crystals) | white/translucent | bipyramidal | clear |
| AS | crystalline | low (<5 crystals) | white/translucent | bipyramidal | clear |
| A9 | crystalline | low (<5 crystals) | white/translucent | bipyramidai | clear |
| A10 | crystalline | low-(<5 crystals) | white/translucent | bipyramidal | clear |
| A11 | crystalline | low (<5 crystals) | white/translucent | bipyramidal | clear |
| A12 | crystalline | low (<5 crystals) | white/translucent | bipyramidal | clear |
| B1 | crystalline | low (<5 crystals) | white/translucent | prisms/trigonal | clear |
| B2 | crystalline | low (<5 crystals) | white/translucent | prisms/trigonal | clear |
| B3 | crystalline | low (<5 crystals) | white/translucent | prisms/trigonal | clear |
| B4 | crystalline | low (<5 crystals) | white/translucent | prisms/trigonal | clear |
| B5 | crystalline | low (<5 crystals) | white/translucent | prisms/trigonal | clear |
| B6 | crystalline | low (<5 crystals) | white/translucent | prisms/trigonal | clear |
| B7 | crystalline | low (<5 crystals) | white/translucent | prisms/trigonal | clear |
| B8 | crystalline | low (<5 crystals) | white/translucent | prisms/trigonal | clear |
| B9 | crystalline | low (<5 crystals) | while/translucent | prisms/trigonal | clear |
| B10 | crystalline | low (<5 crystals) | white/translucent | prisms/trigonal | clear |
| B11 | crystalline | low (<5 crystals) | white/translucent | prisms/trigonal | clear |
| B12 | crystalline | low (<5 crystals) | white/translucent | prisms/trigonal | clear |
| C1 | crystalline | medium (10-30 crystals) | white/opaque | prismatic | clear |
| C2 | crystalline | medium (10-30 crystals) | white/opaque | prismatic | clear |
| C3 | crystalline | medium (10-30 crystals) | white/opaque | prismatic | clear |
| C4 | crystalline | medium (10-30 crystals) | white/opaque | prismatic | clear |
| C5 | crystalline | medium (10-30 crystals) | white/opaque | prismatic | clear |
| C6 | crystalline | medium (10-30 crystals) | white/opaque | prismatic | clear |
| C7 | crystalline | medium (10-30 crystals) | white/opaque | prismatic | clear |
| C8 | crystalline | medium (10-30 crystals) | white/opaque | prismatic | clear |
| C9 | crystalline | medium (10-30 crystals) | white/opaque | prismatic | clear |
| C10 | crystalline | medium (10-30 crystals) | white/opaque | prismatic | clear |
| C11 | crystalline | medium (10-30 crystals) | white/opaque | prismatic | clear |
| C12 | crystalline | medium (<5 crystals) | white/opaque | prismatic | clear |
| D1 | crystalline | high (>30 crystals) | white/translucent | bipyramidal | clear |
| D2 | crystalline | high (>30 crystals) | white/translucent | bipyramidal | clear |
| D3 | crystalline | high (>30 crystals) | white/translucent | bipyramidal | clear |
| D4 | crystalline | high (>30 crystals) | white/translucent | bipyramidal | clear |
| D5 | crystalline | high (>30 crystals) | white/translucent | bipyramidal | clear |
| D6 | crystalline | high (>30 crystals) | white/ translucent | bipyramidal | clear |
| D7 | crystalline | high (>30 crystals) | white/translucent | bipyramidal | clear |
| D8 | crystalline | high (>30 crystals) | white/translucent | bipyramidal | clear |
| D9 | crystalline | high (>30 crystals) | white/translucent | bipyramidal | clear |
| D10 | crystalline | high (>30 crystals) | white/translucent | bipyramidal | clear |
| D11 | crystalline | high (>30 crystals) | white/translucent | bipyramidal | clear |
| D12 | crystalline | high (>30 crystals) | white/translucent | bipyramidal | clear |
| E1 | crystalline | high (>30 crystals) | white/translucent | plates | clear |
| E2 | crystalline | high (>30 crystals) | white/translucent | plates | clear |
| E3 | crystalline | high (>30 crystals) | white/translucent | plates | clear |
| E4 | crystalline | high (>30 crystals) | white/translucent | plates | clear |
| E5 | crystalline | high (>30 crystals) | white/translucent | plates | clear |
| E6 | crystalline | high (>30 crystals) | white/translucent | plates | clear |
| E7 | crystalline | high (>30 crystals) | white/translucent | plates | clear |
| E8 | crystalline | high (>30 crystals) | while/translucent | plates | clear |
| E9 | crystalline | high (>30 crystals) | white/translucent | plates | clear |
| E10 | crystalline | high (>30 crystals) | white/translucent | plates | clear |
| E11 | crystalline | high (>30 crystals) | white/translucent | plates | clear |
| E12 | crystalline | high (>30 crystals) | while/translucent | plates | clear |
| F1 | crystalline | high (>30 crystals) | white/translucent | plates | clear |
| F2 | crystalline | high (>30 crystals) | white/translucent | plates | clear |
| F3 | crystalline | high (>30 crystals) | white/translucent | plates | clear |
| F4 | crystalline | high (>30 crystals) | white/translucent | plates | clear |
| F5 | crystalline | high (>30 crystals) | white/translucent | plates | clear |
| F6 | crystalline | high (>30 crystals) | white/translucent | plates | clear |
| F7 | crystalline | high (>30 crystals) | white/translucent | plates | clear |
| F8 | crystalline | high (>30 crystals) | while/translucent | plates | clear |
| F9 | crystalline | high (>30 crystals) | white/translucent | plates | clear |
| F10 | crystalline | high (>30 crystals) | white/translucent | plates | clear |
| F11 | crystalline | high (>30 crystals) | white/translucent | plates | clear |
| F12 | crystalline | high (>30 crystals) | white/translucent | plates | clear |
| G1 | crystalline | low (<5 crystals) | white/translucent | prisms | clear |
| G2 | crystalline | low (<5 crystals) | white/translucent | prisms | clear |
| G3 | crystalline | low (<5 crystals) | white/translucent | prisms | clear |
| G4 | crystalline | low (<5 crystals) | white/translucent | prisms | clear |
| G5 | crystalline | low (<5 crystals) | white/translucent | prisms | clear |
| G6 | crystalline | low (<5 crystals) | white/translucent | prisms | clear |
| G7 | crystalline | low (<5 crystals) | white/translucent | prisms | clear |
| G8 | crystalline | low (<5 crystals) | white/translucent | prisms | clear |
| G9 | crystalline | low (<5 crystals) | white/translucent | prisms | clear |
| G10 | crystalline | low (<5 crystals) | white/translucent | prisms | clear |
| G11 | crystalline | low (<5 crystals) | white/translucent | prisms | clear |
| G12 | crystalline | low (<5 crystals) | white/translucent | prisms | clear |
| H1 | crystalline | medium (10-30 crystals) | white/translucent | plates | light yellow |
| H2 | crystalline | medium (10-30 crystals) | white/translucent | plates | light yellow |
| H3 | crystalline | medium (10-30 crystals) | white/translucent | plates | light yellow |
| H4 | crystalline | medium (10-30 crystals) | white/translucent | plates | light yellow |
| H5 | crystalline | medium (10-30 crystals) | white/translucent | plates | light yellow |
| H6 | crystalline | medium (10-30 crystals) | white/translucent | plates | light yellow |
| H7 | amorphous | n/a | white/translucent | powder | light yellow |
| H8 | amorphous | n/a | white/translucent | powder | light yellow |
| H9 | amorphous | n/a | white/translucent | powder | light yellow |
| H10 | amorphous | n/a | white/translucent | powder | light yellow |
| H11 | amorphous | n/a | white/translucent | powder | light yellow |
| H12 | amorphous | n/a | white/translucent | powder | lght yellow |

Although the present invention has been described in detail with reference to certain preferred embodiments, other embodiments are possible. Therefore, the spirit and scope of the appended claims should not be limited to the description of the preferred embodiments contained herein. Modifications and variations of the invention described herein will be obvious to those skilled in the art from the foregoing detailed description and such modifications and variations are intended to come within the scope of the appended claims.

A number of references have been cited, the entire disclosures of which are incorporated herein by reference.

1. A system for detecting similarities among a plurality of samples, which comprises
a) a device for obtaining a spectrum for each sample; and
b) a computer configured to analyze each of the spectra and to generate a plurality of sample groups, wherein each sample group corresponds to samples sharing at least one spectral feature and said sample groups are similar polymorphs, hydrates and solvates.

2. The system of claim 1 wherein the device is an infrared spectrometer, near infrared spectrometer, NMR spectrometer, X-ray diffractometer, neutron diffractometer, light microscope, electron microscope, second harmonic generator, circular dichroism spectrometer, linear dichroism spectrometer, differential scanning calorimeter, thermal gravimetric analyzer, or melting point analyzer.

3. The system of claim 1 wherein the device is a Raman spectrometer.

4. The system according to claim 1, wherein said samples are grouped into:
a. wells containing no precipitate;
b. wells with single polymorph;
c. wells with polymorph mixture;
d. wells with amorphous forms of pharmaceutical; and
e. wells with mixtures of: 1) wells containing no precipitate; 2) wells with single polymorph; 3) wells with polymorph mixture; and 4) wells with amorphous forms of pharmaceutical.

5. A system to identify solid-forms of a compound-of-interest, comprising:
a. a computer and an automated distribution mechanism effective to prepare at least 24 samples, each sample comprising the compound-of-interest and one or more components, wherein an amount of the compound-of-interest in each sample is less than about 1 gram;
b. a system effective to process the samples to generate an array comprising at least one solid-form of the compound-of-interest; and
c. a detector to detect the solid-form.

6. The system of claim 5, wherein the detector is a video optical microscope, an image analyzer, an optical microscope, or a polarimeter.

7. The system of claim 5, further comprising an analyzer to analyze the detected solid-form.

8. The system of claim 7, wherein the analyzer is an infrared spectrophotometer, a second harmonic generation optical spectrometer, a mass spectrometer, a nuclear magnetic resonance spectrometer, a near infrared spectrophotometer, a Raman spectrophotometer, an x-ray powder diffractometer, a differential scanning calorimeter, a thermal gravimetric analyzer, a light microscope, or an electron microscope.

9. The system according to claim 5, wherein the system comprises a computer configured to analyze each of the spectra of the samples and to generate a plurality of sample groups, wherein each sample group corresponds to samples sharing at least one spectral feature.
